(19) United States
(12) Patent Application Publication
(10) Pub. No.: US 2026/0369413 A1
(43) Pub. Date: Nov. 19, 2026

(54) METHODS AND SYSTEMS FOR CLASSIFICATION OF A CONDITION USING MASS SPECTROMETRY DATA

(71) Applicant: Bertis Bioscience Incorporated, San Diego, CA (US)

(72) Inventors: Younggon Kim, San Diego, CA (US); Sangtae Kim, San Diego, CA (US)

(21) Appl. No.: 19/115,552

(22) PCT Filed: Sep. 26, 2023
(86) PCT No.: PCT/US2023/033724

§ 371 Date: Mar. 26, 2025

Related U.S. Application Data
(60) Provisional application No. 63/410,054, filed on Sep. 26, 2022.
(60) Provisional application No. 63/531,910, filed on Aug. 10, 2023.

(51) Int. Cl. G16H 50/30; G01N 30/72; G01N 30/86; G01N 33/483; G06N 3/045; G16H 10/40

(52) U.S. Cl. CPC G16H 50/30; G01N 30/724; G01N 30/8693; G01N 33/483; G06N 3/045; G16H 10/40

(57) ABSTRACT

Described herein are methods and systems for characterizing one or more conditions of a subject based on analysis of biological samples obtained from the subject by mass spectrometry.

### CROSS-REFERENCE

This application claims the benefit of U.S. Provisional Application No. 63/410,054, filed Sep. 26, 2022, and U.S. Provisional Application No. 63/531,910, filed Aug. 10, 2023, each of which is incorporated herein by reference in its entirety.

### BACKGROUND

Mass spectrometry (MS) is an analytical technique that measures the mass-to-charge ratio (m/z) of molecules in a sample, providing accurate and specific measurements of molecules even at trace levels. Mass spectrometry is often coupled with liquid chromatography (LC) in biological and clinical studies which provides additional information on molecules based on retention time and can improve signal-to-noise ratios and reduce matrix effects observed by the mass spectrometer. Improvements in mass spectrometers such as high-resolution instruments and faster and more efficient chromatographic methods that have greatly expanded the wealth of information that can be gained through mass spectrometry. Despite the advances made over the past few decades, however, much of the information that can be gained goes unutilized due to the challenging complexity of interpreting mass spectra, particularly when the mass spectrometer is utilizing chromatography. Accordingly, improved methods of analyzing the wealth of data available from mass spectrometry of biological samples are needed.

### SUMMARY

In one aspect, described herein are methods of characterizing a condition of a subject using mass spectrometry data. In some embodiments, the method comprises obtaining one or more raw mass spectra of a sample from the subject collected using a mass spectrometer, wherein the raw mass spectra comprises ion m/z values and intensities, wherein an experimental m/Δm resolving power of the mass spectrometer is about 500-2,000,000 at m/z 200. In some embodiments, the method comprises providing a machine learning model comprising one or more transformers that are trained on a raw mass spectra training dataset for characterization of the condition of the subject. In some embodiments, raw mass spectra are converted to preprocessed mass spectra by an automated algorithm. In some embodiments, the automated algorithm comprises a de-isotoping, a de-charging, or a de-adducting algorithm. In some embodiments, the method comprises using the machine learning model to analyze the one or more raw mass spectra of the sample and output information indicative of at least one condition marker or condition state in the subject.

In some embodiments, the method comprises providing the information to a user via a graphical user interface. In some embodiments, the experimental m/Δm resolving power is about 500-1,000,000 at m/z 200. In some embodiments, the experimental m/Δm resolving power is about 500-30,000 at m/z 200. In some embodiments, the experimental m/Δm resolving power is about 500-5,000 at m/z 200.

In some embodiments, the condition comprises a disease. In some embodiments, the condition comprises an age state of the subject. In some embodiments, the condition comprises a progression-free survival of the subject.

In some embodiments, the machine learning model comprises a plurality of transformers. In some embodiments, the plurality of transformers are arranged in a hierarchy comprising a first and second transformer arranged in a hierarchy such that an output of the first transformer is used as an input of the second transformer. In some embodiments, the one or more raw mass spectra are tokenized prior to submission to the one or more transformers. In some embodiments, the one or more transformers are arranged in a hierarchy with a linear classifier and a random forest aggregator.

In some embodiments, the machine learning model further comprises a linear classifier. In some embodiments, the machine learning model further comprises a neural radiance field. In some embodiments, the machine learning model further comprises a multi-layer neural network. In some embodiments, the machine learning model further comprises a decision tree. In some embodiments, the machine learning model further comprises a support vector machine.

In some embodiments, the one or more raw mass spectra comprise MS/MS spectra. In some embodiments, the one or more raw mass spectra comprise MSⁿ spectra. In some embodiments, the MS/MS or MSⁿ spectra are acquired in a data independent manner.

In some embodiments, the machine learning model is trained with at least 10,000 individual mass spectra per day. In some embodiments, the machine learning model is trained with at least 50,000 individual mass spectra per day. In some embodiments, the machine learning model is trained with at least 100,000 individual mass spectra per day.

In another aspect, described herein are machine-learning based methods of characterizing a condition of a subject. In some embodiments, the method comprises obtaining one or more raw mass spectra of a sample from the subject collected using a mass spectrometer. In some embodiments, the method comprises providing a machine learning model comprising a plurality of transformers that are arranged in a hierarchy and trained on a raw mass spectra training dataset for characterization of the condition. In some embodiments, the method comprises using the machine learning model to analyze the one or more raw mass spectra of the sample and output information indicative of at least one condition or condition state in the subject.

In some embodiments, the hierarchy comprises a first and second transformer in a hierarchy such that an output of the first transformer is used as an input of the second transformer. In some embodiments, the hierarchy further comprises a linear classifier, the linear model being arranged in the hierarchy such that an output of the second transformer is used as an input of the linear classifier.

In some embodiments, the hierarchy further comprises a neural radiance field. In some embodiments, the neural radiance field is arranged in the hierarchy such that an output of the second transformer is used as an input of the neural radiance field. In some embodiments, a neural radiance field replaces one or more of the transformers described herein.

In some embodiments, the hierarchy further comprises a multi-layer neural network. In some embodiments, the multi-layer neural network is arranged in the hierarchy such that an output of the second transformer is used as an input of the multi-layer neural network. In some embodiments, the multi-layer neural network replaces one or more of the transformers described herein.

In some embodiments, the hierarchy further comprises a decision tree, the decision tree being arranged in the hierarchy such that an output of the second transformer is used as an input of the decision tree. In some embodiments, the hierarchy further comprises a support vector machine, the support vector machine being arranged in the hierarchy such that an output of the second transformer is used as an input of the support vector machine. In some embodiments, the first transformer classifies tokenized data based on an MS/MS isolation window. In some embodiments, the classification performed by the first transformer is a summarization of tokenized data from the same MS/MS isolation window.

In some embodiments, the second transformer classifies a vector output of the first transformer based upon a sample identity. In some embodiments, the classification performed by the second transformer is a summarization of data comprising samples obtained from the same subject. In some embodiments, the sample identity comprises an identity of the subject from which the sample was obtained.

In some embodiments, the linear classifier classifies the disease or disease state based on the vector output from the second transformer.

In some embodiments, the raw mass spectra comprise MS/MS spectra that are acquired in a data independent manner. In some embodiments, the machine learning model is trained with at least 10,000 individual mass spectra per day. In some embodiments, the machine learning model is trained with at least 50,000 individual mass spectra per day. In some embodiments, the machine learning model is trained with at least 100,000 individual mass spectra per day.

In some embodiments, the condition comprises a disease. In some embodiments, the condition comprises an age state of the subject. In some embodiments, the condition comprises a progression-free survival of the subject.

In another aspect, described herein are methods of characterizing a condition of a subject using a high throughput trained machine learning model. In some embodiments, the method comprises obtaining one or more raw mass spectra of a sample from the subject collected using a mass spectrometer. In some embodiments, the method comprises providing the machine learning model that is trained on a raw mass spectra training dataset for characterization of the condition, wherein the machine learning model is trained at a rate of at least 10,000 individual raw mass spectra from the training dataset per day. In some embodiments, the method comprises using the machine learning model to analyze the one or more raw mass spectra of the sample and output information indicative of at least one condition in the subject.

In some embodiments, the rate is at least 50,000 individual raw mass spectra from the training set per day. In some embodiments, the rate is at least 100,000 individual raw mass spectra from the training set per day.

In some embodiments, the machine learning model further comprises a linear classifier. In some embodiments, the one or more raw mass spectra comprise MS/MS spectra. In some embodiments, the machine learning model comprises a plurality of transformers. In some embodiments, the plurality of transformers are arranged in a hierarchy comprising a first and second transformer in a hierarchy arranged such that an output of the first transformer is used as an input of the second transformer. In some embodiments, the one or more raw mass spectra are tokenized prior to submission to the one or more transformers. In some embodiments, the condition comprises a disease. In some embodiments, the condition comprises an age state of the subject. In some embodiments, the condition comprises a progression-free survival of the subject.

In some embodiments, the one or more raw mass spectra are tokenized by an MS/MS isolation window and a plurality of m/z values corresponding to detected ions of each of the one or more raw mass spectra. In some embodiments, the one or more raw mass spectra are tokenized such that m/z values with the same unit mass are binned together. In some embodiments, tokenized data comprises multiple entries for the same unit mass. In some embodiments, the multiple entries correspond to separate peaks having the same nominal mass.

In some embodiments, the one or more raw mass spectra are tokenized using large bins (e.g. bins spanning about 1, 0.7, 0.5, or 0.3 mass units). In some embodiments, the one or more raw mass spectra are tokenized using small bins (e.g. bins spanning about 0.1, 0.01, 0.001 or less mass units. In some embodiments, the one or more raw mass spectra are tokenized using uniform bins. In some embodiments, the one or more raw mass spectra are tokenized using non-uniform bins.

In some embodiments, the machine learning model is trained using self-supervised learning. In some embodiments, measuring the sample by mass spectrometry comprises separating components of the sample using liquid chromatography coupled to a mass spectrometer. In some embodiments, a gradient method of the liquid chromatography runs over a period of at least 15 minutes (e.g. about 15, 30, 60, 90, or 180 minutes). In some embodiments, a gradient method of the liquid chromatography runs over a period of about 5 to 10 minutes (e.g. about 5, 7, or 10 minutes).

In some embodiments, the information includes presence or absence of the at least one disease or disease state in the subject. In some embodiments, the at least one disease or disease state comprises cancer. In some embodiments, the cancer comprises pancreatic cancer or ovarian cancer. In some embodiments, the cancer comprises breast cancer. In some embodiments, the cancer comprises prostate cancer. In some embodiments, the cancer comprises lung cancer. In some embodiments, the cancer comprises gallbladder cancer. In some embodiments, the condition comprises a plurality of disease states. In some embodiments, the condition is a disease state, and the disease state comprises a responsiveness of a disease to a therapeutic intervention. In some embodiments, the therapeutic intervention is an immunotherapy (e.g. a CAR-T therapy).

In some embodiments, the information comprises a probability or likelihood of the subject having the at least one disease or disease state. In some embodiments, the information comprises an indication of disease state or disease severity. In some embodiments, the information comprises an indication of disease classification. In some embodiments, the at least one disease or disease state is a cancer and the indication of the disease classification comprises an identification of a cell line genotype or cell line phenotype of the cancer.

In some embodiments, the information is associated with at least one of a proteomic, a lipidomic, or a metabolomic profile of the sample obtained from the subject. In some embodiments, the machine learning model outputs the information without requiring prior domain knowledge relating to at least one of the proteomic, lipidomic, or metabolomic profile. In some embodiments, an accuracy of the information is at least 70%. In some embodiments, an accuracy of the information is at least 80%. In some embodiments, an accuracy of the information is at least 90%. In some embodiments, an accuracy of the information is at least 95%. In some embodiments, an accuracy of the information is at least 99%.

In some embodiments, training the machine learning model to determine a presence or absence of the one or more disease conditions requires no more than about 500 experimental data points. In some embodiments, no more than about 200 experimental data points are required to train the machine learning model. In some embodiments, no more than about 100 experimental data points are required to train the machine learning model.

In some embodiments, an accuracy of the determination is at least about 70%. In some embodiments, the proteomic profile comprises one or more post-translational modifications (PTMs). In some embodiments, the post-translational modifications comprise one or more phosphorylation, acetylation, ubiquitination, glycosylation, or combination of two or more thereof.

In some embodiments, training the machine learning model comprises randomly masking about 1-25% (e.g. 1%, 5%, 10%, 15%, 20%, or 25%) of the training set and adding about 1-10% (e.g. about 1%, 2%, 3%, 4%, 5%, or 10%) noise as a means of self-supervised learning.

In some embodiments, measuring the sample by mass spectrometry comprises separating ions by ion mobility (e.g. by High Field Asymmetric Waveform Ion Mobility Spectrometry (FAIMS) or Drift-tube Ion Mobility Spectrometry) prior to or during acquisition mass spectra. In some embodiments, a mean average percent error of the information is less than about 30% (e.g. less than 30%, 20%, 15%, 10%, 5%, 3%, 2%, or 1%).

In some embodiments, adjacent m/z values are not treated as continuous values during the analysis. In some embodiments, the information comprises identification of one or more signal which is determinative of the presence or absence of a particular condition. In some embodiments, the information comprises identification of one or more signal which is indicative or correlated with a particular state of a particular condition. In some embodiments, the information is used for biomarker discovery.

In some embodiments, the method is capable of being trained at a rate of at least 10 training samples per day (e. g at least 10, 15, 50, 100, 300, 500, or 700 samples per day) when trained using a single GPU or CPU which is no faster in terms of maximum single precision floating point operations per second than an NVidia RTX A6000 GPU equipped with 48 GB of RAM.

In another aspect, described herein are non-transitory computer-readable storage media comprising instructions that, when executed by a processor, causes the processor to perform methods described herein.

In another aspect, described herein are systems configured for characterizing a condition of a subject, the systems comprising: a computer comprising a memory operably coupled to at least one processor; and a module executing in the memory of the computer, the module comprising program code enabled upon execution by the at least one processor of the computer to perform methods described herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings (also “Figure” and “FIG.” herein), of which:

FIG. 1A illustrates an exemplary machine learning architecture for classification of one or more conditions of a subject. Input data from a mass spectrometer is provided to a machine learning model, which comprises a hierarchical transformer arrangement. Raw input data is processed by Transformer L1, which provides its output as input to Transformer L2. L2 output can be further processed by additional steps (shown as optional hierarchy layers) which provide their output as input to a final classifier, or L2 can directly output to the input of the final classifier. The classifier then outputs classification information about the one or more conditions of the subject.

FIG. 1B illustrates an example tokenization of a spectrum. A spectrum having 3 peaks with m/z values 103.009 and 231.068 and 378.136 are converted to a sequence of tokens [231, 103, 378].

FIG. 2 illustrates an example of a machine learning model utilizing hierarchical transformers for classification of the condition of a subject (in this example, identification of disease).

FIG. 3 illustrates self-supervised training of the level 1 transformer used in the example machine learning model shown in FIG. 2.

FIG. 4. illustrates self-supervised training of the level 2 transformer used in the example machine learning model shown in FIG. 2.

FIG. 5. illustrates classification of a condition of a subject from the L2 output.

FIG. 6 illustrates the level 1 encoder peak prediction accuracy and loss progression as training continues.

FIG. 7 illustrates the level 1 encoder adjacent spectrum prediction loss and accuracy progression as training continues.

FIG. 8 illustrates the level 2 encoder spectrum prediction accuracy and loss progression as training continues.

FIG. 9 illustrates the level 2 encoder inter/intra person prediction loss and accuracy progression as training continues, for test and validation sets.

FIG. 10 illustrates that the example training and validation sets produced similar accuracy.

FIG. 11 illustrates example output from an example implementation of the hierarchical transformer scheme shown in FIG. 2.

FIG. 12 illustrates an example of inspecting weights of the top-level linear model of the hierarchical transformer scheme of the example of FIG. 2. Absolute values of the weights indicate the importance of the input feature, I.e., level 2 output. Per isolation window breakdown reveals which isolation window is more important, providing identification of specific condition markers (e.g. biomarkers).

FIG. 13 illustrates inspecting score, i.e., product of level 2 output and weight of the hierarchical transformer scheme of the example of FIG. 2. Score is summed together to get the final classification verdict. By breaking down by window, the window which contributed most to the final score can be identified.

FIG. 14 illustrates inspection of the attention of level 2 transformer of the hierarchical transformer scheme of the example of FIG. 2. Given a specific window, inspecting the attention score of level 2 transformer and check where the model determines to be more important in terms of retention time. The X axis is indicative of time.

FIG. 15 shows a computer system that is programmed or otherwise configured to implement methods provided herein.

FIG. 16 illustrates an alternate exemplary machine learning architecture for classification of one or more conditions of a subject. Input data from a mass spectrometer is provided to a machine learning model, which comprises a hierarchical transformer arrangement. Raw input data is processed by Transformer L1, which provides its output as input to a linear classifier (or to additional process steps (shown as optional hierarchy layers) which input to the linear classifier), which is aggregated by a random forest model which outputs classification information about the one or more conditions of the subject.

FIG. 17 illustrates a more detailed implementation of the example machine learning model depicted in FIG. 16.

FIG. 18 illustrates conversion of a spectrum into a sequence useful for training example models described herein.

FIG. 19 illustrates a conceptual analogy between sentence and spectrum pre-training of models described herein.

FIG. 20A illustrates example results from a test case of an exemplary machine learning model described herein for Protein P01861.

FIG. 20B illustrates example results from a test case of an exemplary machine learning model described herein for Protein P08519.

FIG. 21 illustrates example results from a test case of an exemplary machine learning model described herein for Protein P01861.

FIG. 22 illustrates the accuracy of an exemplary machine learning model described herein in various test cases.

FIG. 23 illustrates the accuracy of an exemplary machine learning model described herein in alternate test cases described herein.

### DETAILED DESCRIPTION

While various embodiments of the present disclosure have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

As used herein, the terms “MS”, “mass spec” and “mass spectrometer” are used interchangeably to refer to a device which separates ions in time, space, or both based on a mass to charge ratio (m/z) of the ions.

Recognized herein is the need for systems and methods for characterizing a condition of a subject using machine learning techniques coupled with mass spectrometry data of a sample obtained from the subject.

Metabolomics, lipidomics, and/or proteomics can provide key insights into the health and functionality of a biological system. These tools can provide information useful for assessing the health status of human or animal subjects, as select metabolites, lipids, and proteins serve as biomarkers for various states of disease, malnutrition, or cellular dysfunction. For example, conditions such as diabetes mellitus, metabolic syndrome, renal failure, and hepatic failure present with biomarkers recognizable in blood or urine. Other cellular dysfunctions, such as various cancers, provide biomarker signatures that enable early detection of disease or monitoring of disease progression. Thus, analysis of biomarkers is of key utility for the fields of medical and veterinary science.

Analysis of biological samples by mass spectrometry provides access to the wealth of information provided by metabolomics, lipidomics, proteomics. Experiments in biological mass spectrometry can start with a neutral liquid sample and ends with the detection of a charged gas phase ion.

One aspect of the present disclosure provides a method comprising: applying mass spectrometry (MS) to a sample and using a trained machine learning model to determine information about one or more condition of a sample obtained from a subject.

In another aspect, described herein are non-transitory computer-readable storage medium comprising a set of instructions for executing a method described herein. In some embodiments, the machine learning model is selected from logistic regression, ada boost classifier, extra trees classifier, extreme gradient boosting, gaussian process classifier, gradient boosting classifier, K-nearest neighbor, light gradient boosting, linear discriminant analysis, multi-level perceptron, naïve Bayes, quadratic discriminant analysis, random forest classifier, ridge classifier, SVM (linear and radial kernels), fully connected neural network, or a deep neural network.

One aspect of the present disclosure provides a system for classification of a condition of a subject based on a sample obtained from the subject comprising: a computing unit operably coupled to a mass spec (MS) machine.

In some embodiments a sample obtained from a subject can be a cell, a tissue, a urine, a fecal matter, a blood, a blood plasma, a mucus, a saliva, a blood serum, a cerebrospinal fluid, or a cyst fluid.

Samples may be analyzed using chromatography. In some cases, the chromatography comprises liquid chromatography (LC). Chromatography generally comprises a laboratory technique for the separation of a mixture into its components. A mixture can be dissolved into a mobile phase, which can be carried through a system, such as a column, comprising a fixed stationary phase. The components within the mobile phase may have different affinities to the stationary phase, resulting in different retention times depending on these affinities. As a result, separation of components in the mixture is achieved.

The separated components from chromatography may be analyzed using a mass spectrometer (MS). The LC output may be passed to an MS either directly or indirectly. Mass spectrometric analysis generally refers to measuring the mass-to-charge ratio of ions (e.g., m/z), resulting in a mass spectrum. The mass spectrum comprises a plot of intensity as a function of mass-to-charge ratio. The mass spectrum may be used to determine elemental or isotopic signatures in a sample, as well as the masses of the components (e.g., particles or molecules) in the mixture. This may be used to determine a chemical identity or structure of the components in the mixture.

In some cases, one or more acquisition parameters is programmed in the MS. In some instances, the one or more acquisition parameters comprises, for example, the one or more mass acquisition windows, one or more acquisition times for the one or more mass acquisition windows, one or more resolutions for the one or more mass acquisition windows, one or more gain settings for the one or more acquisition windows, one or more ionization polarity settings for the one or more mass acquisition windows, one or more mass resolutions for the one or more mass acquisition windows, or any combination thereof. In some cases, the MS is a high-resolution mass spectrometer. In some cases, the MS is a low-resolution mass spectrometer. In some instances, the high-resolution mass spectrometer has a mass accuracy is less than or equal to 75 ppm, less than or equal to 30 ppm, less than or equal to 15 ppm, less than or equal to 10 ppm, or less than or equal to 5 ppm.

The output signal from the MS can comprise an intensity value, a mass-to-charge ratio, or a combination thereof. In some cases, the output signal from the MS comprises raw, unprocessed MS data. In some cases, the output signal comprises a first signal indicating an intensity value or a mass-to-charge ratio of one or more analytes. In some cases, the output signal comprises a second signal indicating an intensity value or a mass-to-charge ratio of one or more calibrators. In some cases, the output signal comprises the first signal and the second signal. In some instances, the output signal comprises the peak signal intensity obtained for an exact isotopic mass for each of the one or more analytes or one or more calibrators of known molecular weight. In some instances, the output signal comprises combined signals corresponding to one or more mass adducts for the one or more analytes. In some examples, the output signal for the one or more analytes is obtained by calculating the sum of the adduct signals for 1, 2, 3, 4, 5, 6, 7, 8, 9 or 10 analyte adducts. In some cases, the analyte adducts correspond to the proton, sodium, potassium, calcium, magnesium, ammonium, nitrate, sulfate, phosphate, acetate, citrate, or formate adducts.

In some embodiments, the MS is a tandem MS (MS/MS). In MS/MS mode, a tandem MS can be operated such that ions passing through a first mass spec are activated and the m/z of the activated ions after a fixed amount of time. The second MS produces a mass spectrum comprising the activated ions and any fragments thereof produced during or after the ion activation. Isolation windows can be selected to determine which ions are subjected to activation and subsequent analysis. In a data independent acquisition mode, the isolation windows are fixed by the operator. In a data dependent acquisition mode, the isolation windows can be adjusted during the course of data acquisition, for example to activate the most or least abundant ions in a spectrum for subsequent analysis of fragmentation.

In some cases, the LC-MS method provided herein is optimized for performance on a subset of cellular analytes. In some cases, the LC-MS methods provided herein ionizes in both positive and negative modes. In some cases, the LC-MS method provided herein ionize analytes as molecular ions. In some cases, an ion mobility separation is performed prior to, or during mass spectrometry analysis.

The output signal from the MS (e.g., mass spectrum comprising intensity value, mass-to-charge ratio, and/or timing information; or tandem mass spectra) may be processed by a signal processing module. The input to the signal processing module can comprise an input signal comprising an intensity value, a mass-to-charge ratio, timing information, or a combination thereof from the MS.

In some cases, the input to the signal processing module comprises raw or unprocessed MS data. In some cases the input is an MZML file comprising the raw, unprocessed MS data. In some cases, the input comprises preprocessed MS data. Preprocessing MS data may comprise data cleaning, data transformation, data reduction, or any combination thereof. In some cases, data cleaning comprises cleaning missing data (e.g., fill in or ignore missing values), noisy data (e.g., binning, regression, clustering, etc.), or a combination thereof. In some cases, data transformation comprises standardization, normalization, attribute selection, discretization, hierarchy generation, or any combination thereof. In some cases, data reduction comprises data aggregation, attribute subset selection, numerosity reduction, dimensionality reduction, or any combination thereof. In some cases, the MS data is preprocessed prior to the signal processing module. In some cases, the MS data is preprocessed in the signal processing module. The signal processing module can comprise a machine learning model. The machine learning model can be trained on MS data. The machine learning model may be a trained machine learning algorithm. The trained machine learning model may be used to determine information about a condition of a sample obtained from a subject.

A machine learning model can comprise a supervised, semi-supervised, unsupervised, or self-supervised machine learning model. In some cases, the one or more ML approaches perform classification or clustering of the MS data. In some examples, the machine learning approach comprises a classical machine learning method, such as, but not limited to, support vector machine (SVM) (e.g., one-class SVM, linear or radial kernels, etc.), K-nearest neighbor (KNN), isolation forest, random forest, logistic regression, AdaBoost classifier, extra trees classifier, extreme gradient boosting, gaussian process classifier, gradient boosting classifier, light gradient boosting, linear discriminant analysis, naïve Bayes, quadratic discriminant analysis, ridge classifier, or any combination thereof. In some examples, the machine learning approach comprises a deep leaning method (e.g., deep neural network (DNN)), such as, but not limited to a fully-connected network, convolutional neural network (CNN) (e.g., one-class CNN), recurrent neural network (RNN), transformer, graph neural network (GNN), convolutional graph neural network (CGNN), multi-level perceptron (MLP), or any combination thereof.

In some embodiments, a classical ML method comprises one or more algorithms that learns from existing observations (i.e., known features) to predict outputs. In some embodiments, the one or more algorithms perform clustering of data. In some examples, the classical ML algorithms for clustering comprise K-means clustering, mean-shift clustering, density-based spatial clustering of applications with noise (DBSCAN), expectation-maximization (EM) clustering (e.g., using Gaussian mixture models (GMM)), agglomerative hierarchical clustering, or any combination thereof. In some embodiments, the one or more algorithms perform classification of data. In some examples, the classical ML algorithms for classification comprise logistic regression, naïve Bayes, KNN, random forest, isolation forest, decision trees, gradient boosting, support vector machine (SVM), or any combination thereof. In some examples, the SVM comprises a one-class SMV or a multi-class SVM.

In some embodiments, the deep learning method comprises one or more algorithms that learns by extracting new features to predict outputs. In some embodiments, the deep learning method comprises one or more layers. In some embodiments, the deep learning method comprises a neural network (e.g., DNN comprising more than one layer). In some embodiments, the output from a given node is passed on as input to another node. The nodes in the network generally comprise input units in an input layer, hidden units in one or more hidden layers, output units in an output layer, or a combination thereof. In some embodiments, an input node is connected to one or more hidden units. In some embodiments, one or more hidden units is connected to an output unit. The nodes can generally take in input through the input units and generate an output from the output units using an activation function. In some embodiments, the input or output comprises a tensor, a matrix, a vector, an array, or a scalar. In some embodiments, the activation function is a Rectified Linear Unit (ReLU) activation function, Gaussian Error Linear Unit (GeLU), a sigmoid activation function, a hyperbolic tangent activation function, or a Softmax activation function.

The connections between nodes further comprise weights for adjusting input data to a given node (i.e., to activate input data or deactivate input data). In some embodiments, the weights are learned by the neural network. In some embodiments, the neural network is trained to learn weights using gradient-based optimizations. In some embodiments, the gradient-based optimization comprises one or more loss functions. In some embodiments, the gradient-based optimization is gradient descent, conjugate gradient descent, stochastic gradient descent, or any variation thereof (e.g., adaptive moment estimation (Adam)). In some further embodiments, the gradient in the gradient-based optimization is computed using backpropagation. In some embodiments, the nodes are organized into graphs to generate a network (e.g., graph neural networks). In some embodiments, the nodes are organized into one or more layers to generate a network (e.g., feed forward neural networks, convolutional neural networks (CNNs), recurrent neural networks (RNNs), etc.). In some embodiments, the CNN comprises a one-class CNN or a multi-class CNN.

In some embodiments, the neural network comprises one or more recurrent layers. In some embodiments, the one or more recurrent layers are one or more long short-term memory (LSTM) layers or gated recurrent units (GRUs). In some embodiments, the one or more recurrent layers perform sequential data classification and clustering in which the data ordering is considered (e.g., time series data). In such embodiments, future predictions are made by the one or more recurrent layers according to the sequence of past events. In some embodiments, the recurrent layer retains important information, while selectively removing what is not essential to the classification.

In some embodiments, the neural network comprise one or more convolutional layers. In some embodiments, the input and the output are a tensor representing variables or attributes in a data set (e.g., features), which may be referred to as a feature map (or activation map). In such embodiments, the one or more convolutional layers are referred to as a feature extraction phase. In some embodiments, the convolutions are one dimensional (1D) convolutions, two dimensional (2D) convolutions, three dimensional (3D) convolutions, or any combination thereof. In further embodiments, the convolutions are 1D transpose convolutions, 2D transpose convolutions, 3D transpose convolutions, or any combination thereof.

The layers in a neural network can further comprise one or more pooling layers before or after a convolutional layer. In some embodiments, the one or more pooling layers reduces the dimensionality of a feature map using filters that summarize regions of a matrix. In some embodiments, this down samples the number of outputs, and thus reduces the parameters and computational resources needed for the neural network. In some embodiments, the one or more pooling layers comprises max pooling, min pooling, average pooling, global pooling, norm pooling, or a combination thereof. In some embodiments, max pooling reduces the dimensionality of the data by taking only the maximums values in the region of the matrix. In some embodiments, this helps capture the most significant one or more features. In some embodiments, the one or more pooling layers is one dimensional (1D), two dimensional (2D), three dimensional (3D), or any combination thereof.

The neural network can further comprise of one or more flattening layers, which can flatten the input to be passed on to the next layer. In some embodiments, a input (e.g., feature map) is flattened by reducing the input to a one-dimensional array. In some embodiments, the flattened inputs can be used to output a classification of an object. In some embodiments, the classification comprises a binary classification or multi-class classification of visual data (e.g., images, videos, etc.) or non-visual data (e.g., measurements, audio, text, etc.). In some embodiments, the classification comprises binary classification of an image (e.g., cat or dog). In some embodiments, the classification comprises multi-class classification of a text (e.g., identifying hand-written digits)). In some embodiments, the classification comprises binary classification of a measurement. In some examples, the binary classification of a measurement comprises a classification of a system's performance using the physical measurements described herein (e.g., normal or abnormal, normal or anormal).

The neural networks can further comprise of one or more dropout layers. In some embodiments, the dropout layers are used during training of the neural network (e.g., to perform binary or multi-class classifications). In some embodiments, the one or more dropout layers randomly set some weights as 0 (e.g., about 10%, 20%, 30%, 40%, 50%, 60%, 70%, 80% of weights). In some embodiments, the setting some weights as 0 also sets the corresponding elements in the feature map as 0. In some embodiments, the one or more dropout layers can be used to avoid the neural network from overfitting.

The neural network can further comprise one or more dense layers, which comprises a fully connected network. In some embodiments, information is passed through a fully connected network to generate a predicted classification of an object. In some embodiments, the error associated with the predicted classification of the object is also calculated. In some embodiments, the error is backpropagated to improve the prediction. In some embodiments, the one or more dense layers comprises a Softmax activation function. In some embodiments, the Softmax activation function converts a vector of numbers to a vector of probabilities. In some embodiments, these probabilities are subsequently used in classifications, such as classifications of a type or class of a molecule (e.g., calibrator or analyte) as described herein.

In some cases, the model comprises multi-modality models. In some cases, multi-modality models can be extremely powerful. Different modalities provide supportive, complementary or even completely orthogonal signals to the model. Multi-modality models allow the model to be use for a variety of downstream tasks that might benefit from some or all of the input modalities. Intermediate features and terminal embeddings from each model are fused. The fused representation is then used to train subsequent models for various tasks including regression, classification, generation and dimensionality reduction. The entire network and sub-models can be fine-tuned for specific tasks or the sub-models can be frozen and only the heads trained and/or finetuned. The modularity offers the flexibility of interchanging a sub-model by higher performing models as they become available or designed. Sub-models can take any form, such as, but not limited to, CNN, Transformer, MLP, etc. Each module can then be used to generate embeddings for new unseen data that can then be used for downstream tasks.

The training data may be designed based on one or more considerations. Considerations may comprise, by way of non-limiting example, effective LC separation of the broadest range of analytes, instrumental conditions for collective sensitivity of all analytes (ionization mode, RT, extracted ion chromatogram for each analyte), inherent range (high and low) of instrument detection (for each analyte), resolving power of the mass spectrometer, length of time between injections (acquisition and column equilibration), stability and reproducibility over long acquisition times, MS/MS parameters (e.g. isolation windows for data independent analysis (DIA)), and/or use of spiked-in non-endogenous QC analytes to demarcate between sample issues and instrument issues.

For example, training data may comprise raw spectra comprising data on a plurality of samples collected from populations of subjects with one or more known conditions. The instruments can comprise two or more different mass spectrometer types (e.g. ion trap, orbitrap, FT-ICR, time-of-flight (ToF), or QQQ-time-of-flight (QTOF) mass spectrometers). The instruments can comprise two or more different mass spectrometers of the same type. Inclusion of the one or more design considerations in building the training set can produce a model which is capable of accurately classifying a sample obtained from a subject having an unknown condition based on analysis of MS data obtained from the sample.

In some cases, a run list of samples is provided by a user interface, for example to facilitate construction of the training set using an MS or LC-MS equipped with an autosampler. In some instances, the user interface comprises information such as sample plate positions, blank positions, number of drawers, number of slots per drawer, columns to run, blank plate number of wells, number of injections, plates between calibration curves, maximum blank well reuse, injection volume, blank frequency, etc.

In some embodiments, the mass accuracy is less than or equal to 75 ppm, less than or equal to 30 ppm, less than or equal to 15 ppm, less than or equal to 10 ppm, or less than or equal to 5 ppm.

In some embodiments, methods described herein do not require exact mass (e.g. data from a low resolution mass spectrometer such as a conventional ion trap may be used) in order to provide classification of a condition of a subject based on analysis of a sample obtained from the subject.

In some cases, training data can comprise multi-modal foundation models. The foundation models can be trained using meta data inputs comprising MS1 and/or MS2 spectra. In some instances, the underlying architecture is modality agnostic. For example, a modality-agnostic foundational model may be trained to understand mass spectra indifferently to whether the spectra are acquired in MS1, MS2, Multiple Reaction Monitoring (MRM), Data-Independent Acquisition (DIA), Data Dependent Acquisition (DDA), MSⁿ mode, or combinations thereof.

In some embodiments, a multi-modal modal or mode-agnostic model described herein can translate from one modality to another based at least in part on data describing a joint space between two or more modalities.

In some embodiments, training a model described herein using inputs from a plurality of different modalities reduces or eliminates the need for labeling of training and/or sample data. For example, training using a combination of MS1 and MS2 data can reduce or eliminate the need for labeled datasets for a particular downstream application (such as biomarker discovery and/or disease classification). In some embodiments, use of a multi-modal training regime can significantly reduce a number of empirical data points needed to make a disease classification or discover a biomarker. Such embodiments are particularly advantageous for classification of rare or complex conditions where the availability of controlled empirical data is limited and/or non-existent.

In some embodiments, multi-modal models allow utilization of mass spectrometry measurements of less than 150 clinical samples (e.g. as few as 10 to 20 samples) to provide accurate characterization of a disease or condition.

In some embodiments, foundational multi-modal models can be fine-tuned using a small number of data points, for example, to train for specific characterizations such as identification of gene labels and/or metabolites.

In some embodiments, multi-modal models are trained using m/z peaks with raw intensity values from a plurality of mass spectrometer operating modes to form a vocabulary of the model. In some cases, continuous values can be converted to discreet inputs, representing intensity, m/z and/or mode of acquisition. In some cases, chromatographic information can be included to further refine the models, or intentionally excluded to produce a model which is LC agnostic.

In some embodiments, training data comprises millions of paired m/z, intensity data points. In certain embodiments, the precision of data points is compressed by breaking discreet values into the first three and last three digits to reduce the dimensionality of the training set (e.g. from millions of data points to about 1000 different vectors).

### Computer Systems

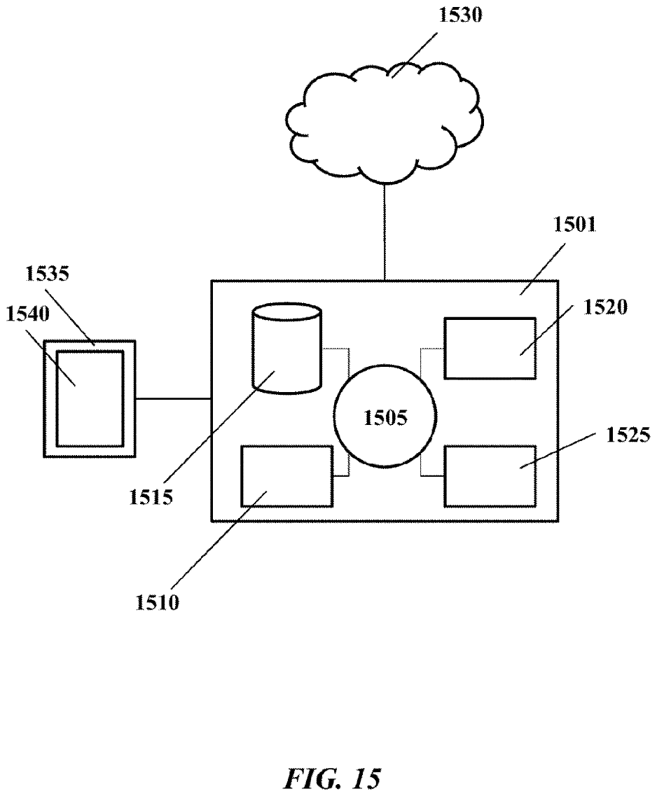

The present disclosure provides computer systems that are programmed to implement methods of the disclosure. FIG. 15 shows a computer system 1501 that is programmed or otherwise configured to characterize a condition of a subject using mass spectrometry data obtained by analyzing a sample collected from the subject. The computer system 1501 can regulate various aspects of the machine-learning based methods of the present disclosure, such as, for example, providing a model which is capable of providing output information indicative of at least one condition marker or condition state in the subject. The computer system 1501 can be an electronic device of a user or a computer system that is remotely located with respect to the electronic device. The electronic device can be a mobile electronic device.

The computer system 1501 includes a central processing unit (CPU, also “processor” and “computer processor” herein) 1505, which can be a single core or multi core processor, or a plurality of processors for parallel processing. The computer system 1501 also includes memory or memory location 1510 (e.g., random-access memory, read-only memory, flash memory), electronic storage unit 1515 (e.g., hard disk), communication interface 1520 (e.g., network adapter) for communicating with one or more other systems, and peripheral devices 1525, such as cache, other memory, data storage and/or electronic display adapters. The memory 1510, storage unit 1515, interface 1520 and peripheral devices 1525 are in communication with the CPU 1505 through a communication bus (solid lines), such as a motherboard. The storage unit 1515 can be a data storage unit (or data repository) for storing data. The computer system 1501 can be operatively coupled to a computer network (“network”) 1530 with the aid of the communication interface 1520. The network 1530 can be the Internet, an internet and/or extranet, or an intranet and/or extranet that is in communication with the Internet. The network 1530 in some cases is a telecommunication and/or data network. The network 1530 can include one or more computer servers, which can enable distributed computing, such as cloud computing. The network 1530, in some cases with the aid of the computer system 1501, can implement a peer-to-peer network, which may enable devices coupled to the computer system 1501 to behave as a client or a server.

The CPU 1505 can execute a sequence of machine-readable instructions, which can be embodied in a program or software. The instructions may be stored in a memory location, such as the memory 1510. The instructions can be directed to the CPU 1505, which can subsequently program or otherwise configure the CPU 1505 to implement methods of the present disclosure. Examples of operations performed by the CPU 1505 can include fetch, decode, execute, and writeback.

The CPU 1505 can be part of a circuit, such as an integrated circuit. One or more other components of the system 1501 can be included in the circuit. In some cases, the circuit is an application specific integrated circuit (ASIC).

The storage unit 1515 can store files, such as drivers, libraries and saved programs. The storage unit 1515 can store user data, e.g., user preferences and user programs. The computer system 1501 in some cases can include one or more additional data storage units that are external to the computer system 1501, such as located on a remote server that is in communication with the computer system 1501 through an intranet or the Internet.

The computer system 1501 can communicate with one or more remote computer systems through the network 1530. For instance, the computer system 1501 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1501 via the network 1530.

Methods as described herein can be implemented by way of machine (e.g., computer processor) executable code stored on an electronic storage location of the computer system 1501, such as, for example, on the memory 1510 or electronic storage unit 1515. The machine executable or machine readable code can be provided in the form of software. During use, the code can be executed by the processor 1505. In some cases, the code can be retrieved from the storage unit 1515 and stored on the memory 1510 for ready access by the processor 1505. In some situations, the electronic storage unit 1515 can be precluded, and machine-executable instructions are stored on memory 1510.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1501 can include or be in communication with an electronic display 1535 that comprises a user interface (UI) 1540 for providing, for example, information concerning a condition of a sample obtained from a subject. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1505. The algorithm can, for example, be configured to perform any of the methods described herein.

### EXAMPLES

### Example 1: Direct Disease Classification From Raw Mass-Spectrometry Data Using Self-Supervised Deep Learning

Deep learning has made great strides in many areas, but in proteomics, the adaptation has been limited to a small number of applications, such as prediction of chromatographic retention time and product ion intensities for specific ions. An untapped potential of deep learning was demonstrated by building a model that classifies patients into groups of cancer patients and groups of normal subjects by directly analyzing data-independent acquisition (DIA) data, without needing any prior proteomics knowledge.

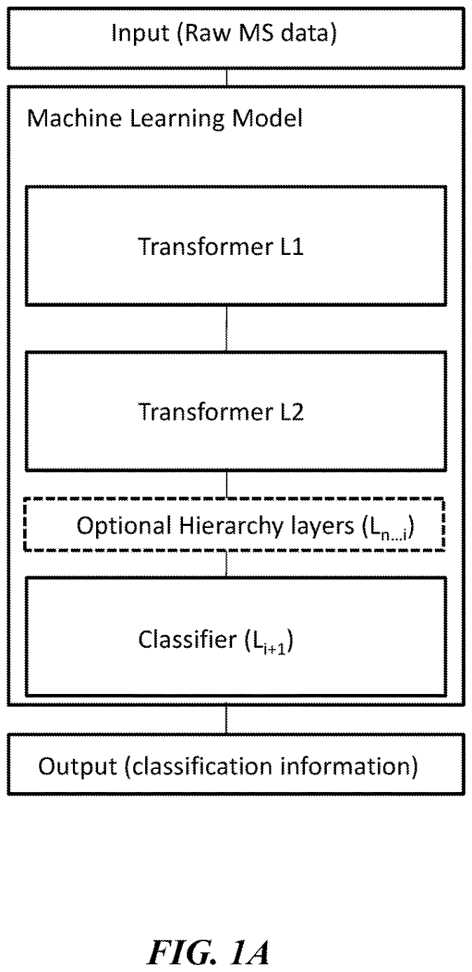

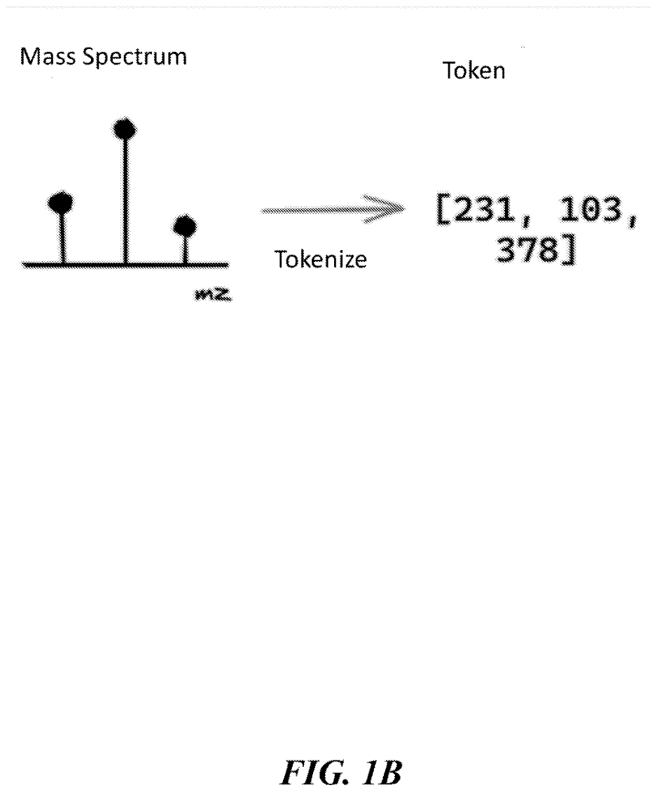

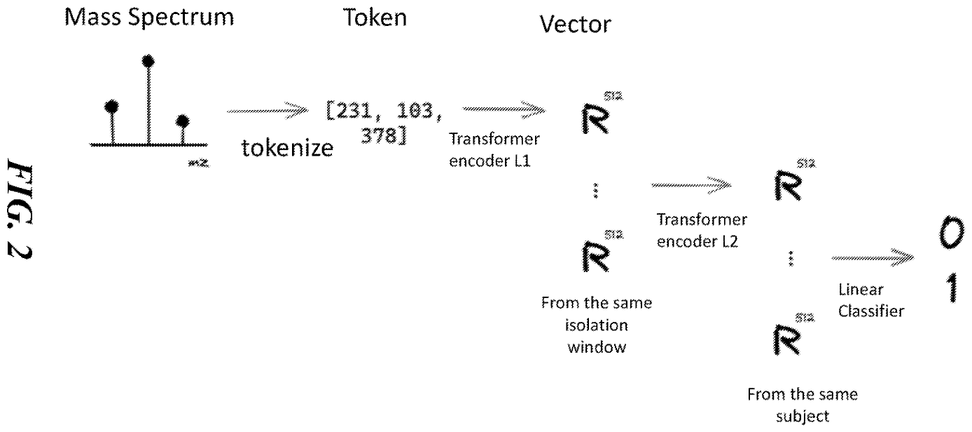

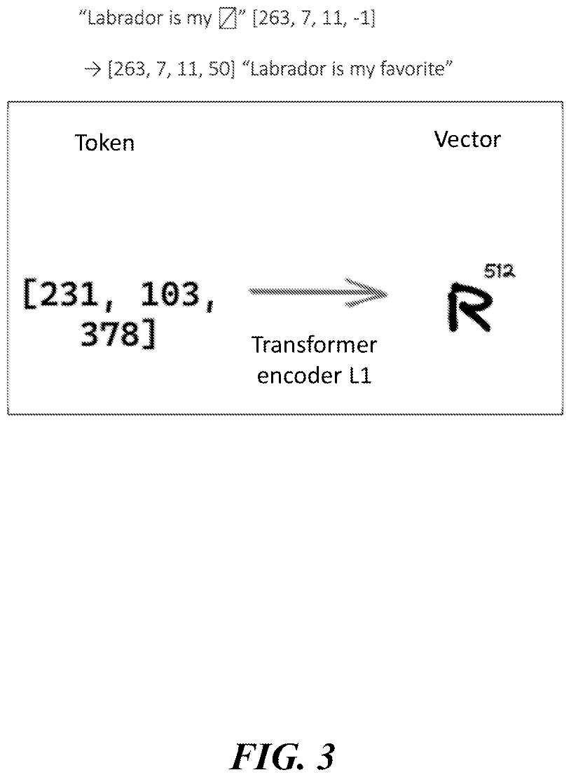

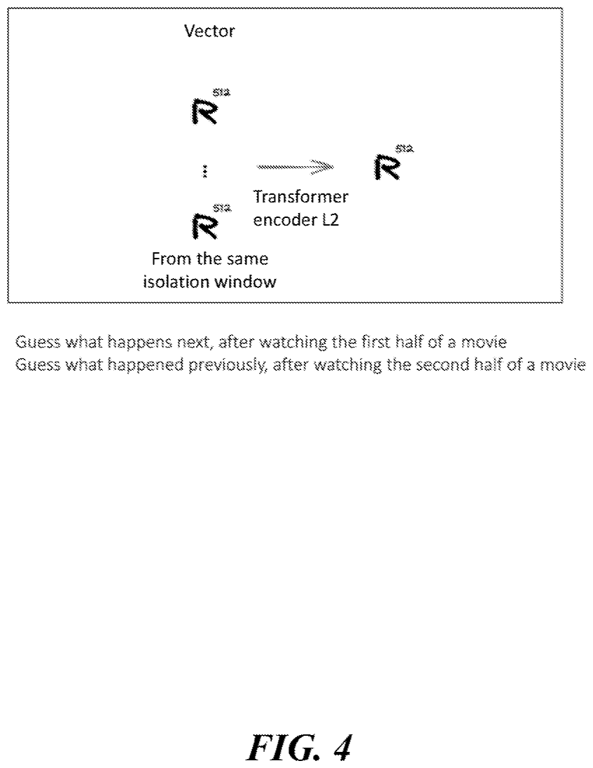

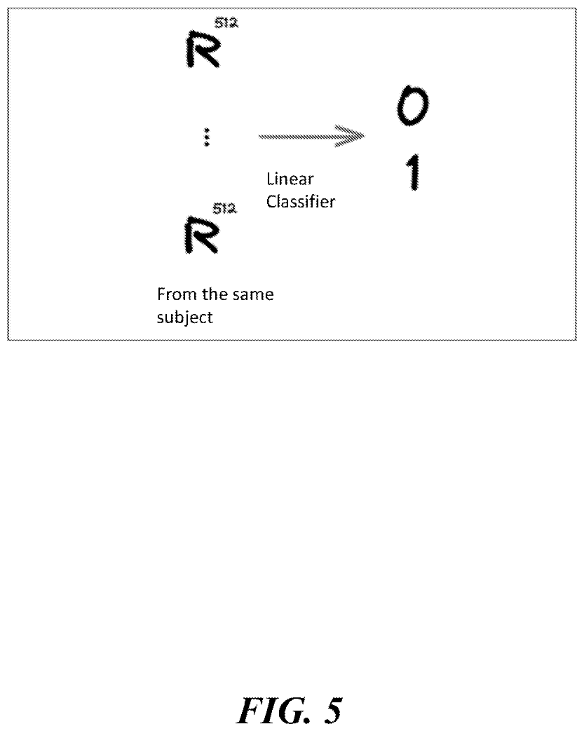

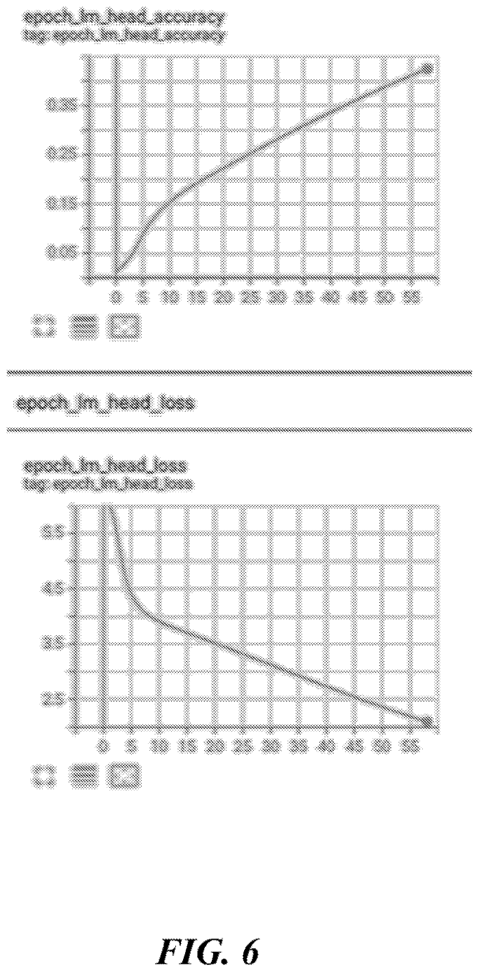

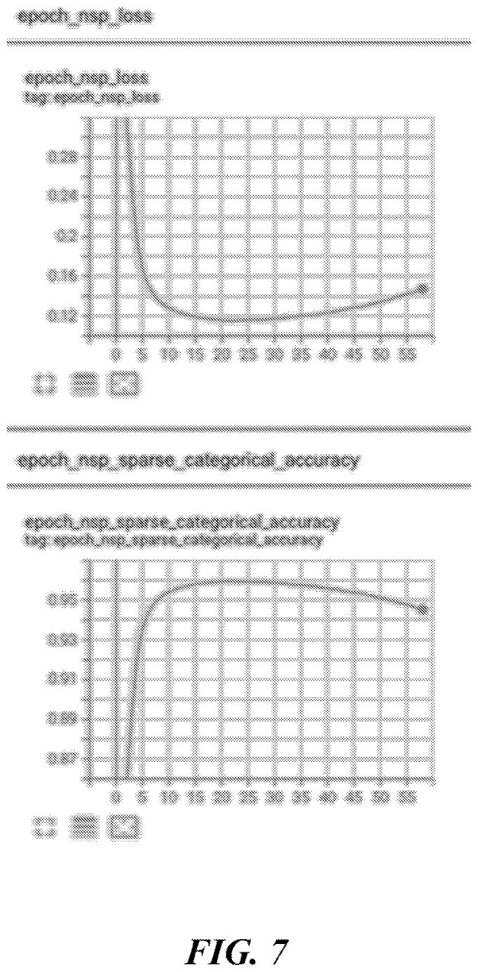

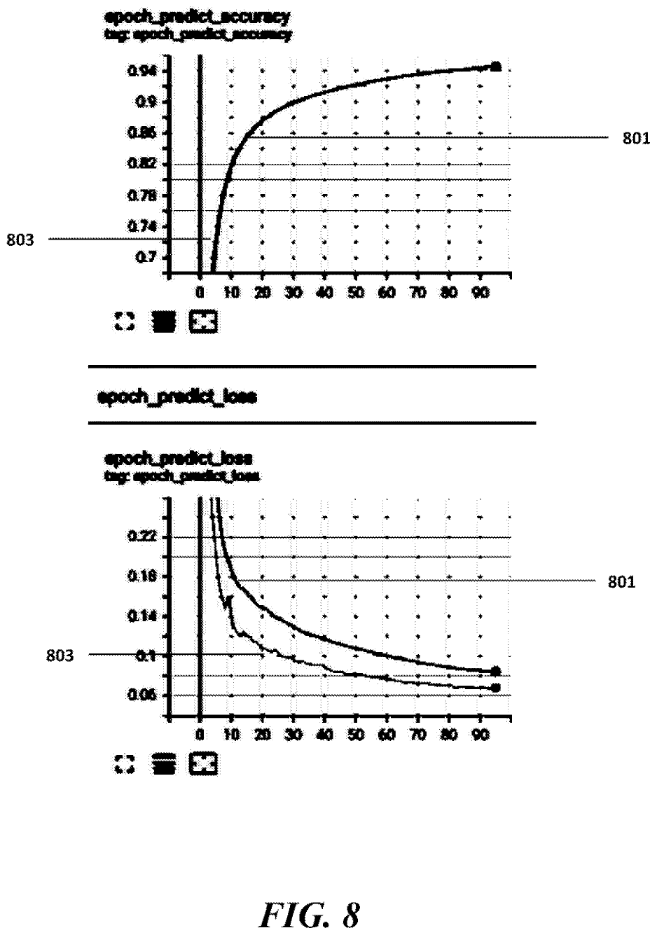

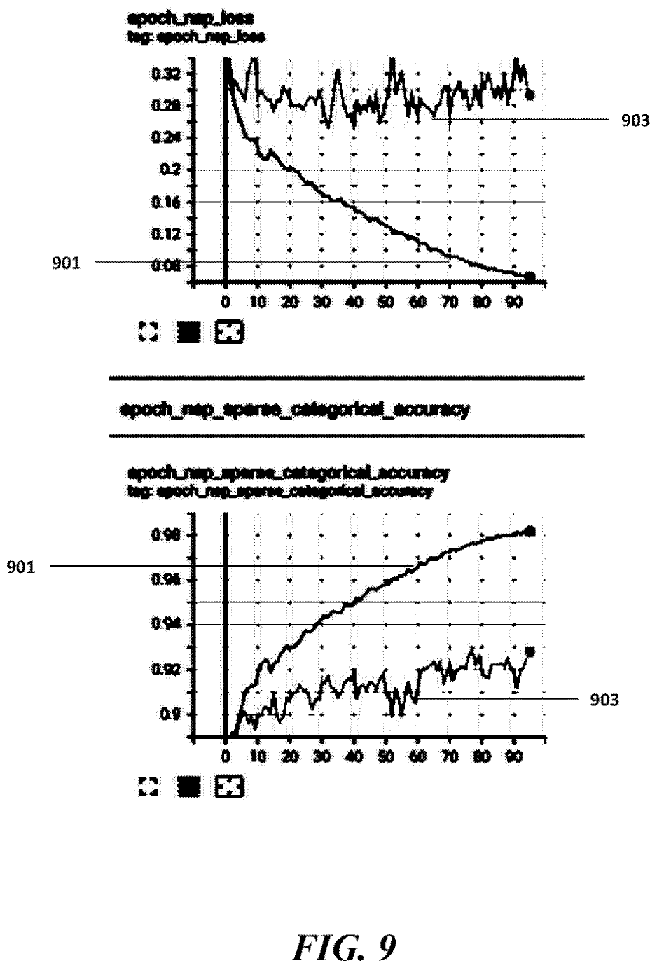

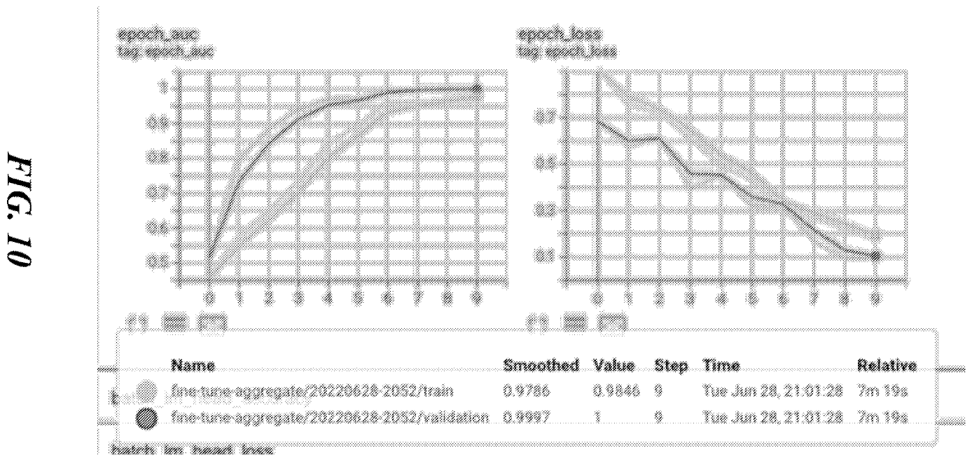

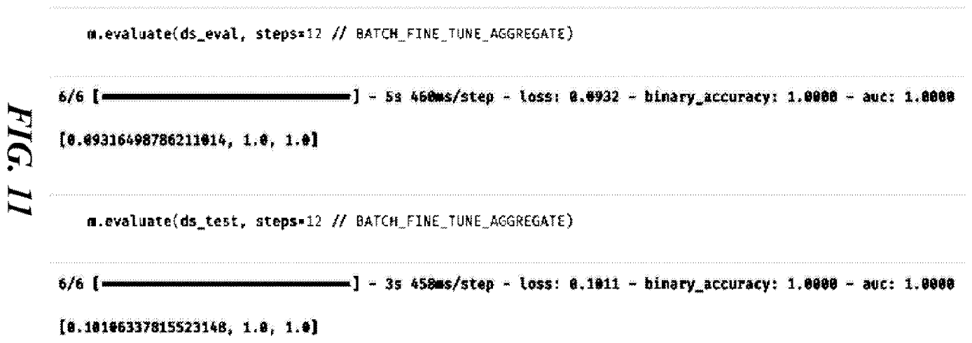

Transformer encoders were used for encoding DIA data. To facilitate the processes of a large data set, the encoders were laid out in a hierarchy according to the arrangements described in FIGS. 1A, and 2. The level-1 transformer encoded each MS/MS spectrum, and the level-2 transformer encoded a sequence of level-1 outputs. Both encoders were trained in a self-supervised fashion—learning the distribution itself without externally added labels. In the self-supervised training, novel optimization objectives were added on top of the typical objective of predicting hidden input. After training each level in sequence, the top-level classifier was fine-tuned along with the level-2 transformer. The labels used in the final fine-tune step are the only external information injected to the model.

This method was tested on two DIA datasets—ovarian cancer with 157 samples and pancreatic cancer with 118 samples, each of which contained ~50% of healthy samples as control. The datasets were split into 80% training, 10% evaluation and 10% test sets. For the split, both models achieved an area under curve of 1.0 in classifying the cancer and normal samples.

The results demonstrate that deep learning architectures and training regimes are beneficial to mass spectrometry-based proteomics and/or for classification of conditions of a subject (such as cancer). Minimal domain knowledge was used to develop the model which is capable of accurately distinguishing samples obtained from subjects with cancer from cancer-free subjects, demonstrating that the method can be extended to other mass spectrometry-based omics technologies (e.g., metabolomics and lipidomics) and their integrations. This work paves a way toward making sense of underutilized information, such as post-translational modifications and discovery of new biomarkers.

### Example 2—Spectrum is All You Need (SAN )

A machine-learning model, termed Spectrum is All you Need (SAN), was designed which aims to analyze MS data using deep learning, with minimal domain knowledge. A transformer, which is widely used in natural language processing (NLP), computer vision, speech processing, as well as in bioinformatics, was used as the main engine of the architecture. The use of transformer resulted in several interesting design decisions:
Tokenization: each spectrum was converted to a sequence of tokens, similar to a sentence being converted to tokens in NLP.
Hierarchy: Transformers conventionally struggle with a long data sequences. Accordingly, input data was split and fed to a hierarchy of models to reduce the size of the data sequence each transformer was required to handle (see FIG. 1A).
Self-supervised training: Transformers ordinarily require a lot of examples to train properly. Due to the wealth of data available by mass spectrometric analysis of samples, particularly when paired with additional separations (e.g. chromatography, ion mobility, etc.), even a single sample comprises a very large data set. Accordingly, self-supervised training was used to decrease the number of experimental data points required. Several self-supervised objectives were devised and used to reduce the total number of training points required to build an accurate model.
Tokenization: A unique tokenization procedure was utilized. Mass spectra generally include a set of peaks where each peak has a mass-to-charge ratio (m/z) and an intensity. Each tandem mass (MS/MS) spectrum was converted to a sequence of tokens by, first sorting all the peaks in the decreasing order of their intensities (e.g. most intense peaks first), and then converting peaks to token ids by rounding up the m/z value to the closest integer after multiplying a scaling constant (0.9995 by default). Using this particular tokenization scheme the peaks whose m/z values were close together were assigned the same token id, indicating that high-resolution isn't necessarily needed to provide accurate classification. Each token then became a categorical variable which does not carry any explicit information about the m/z value. All relationships among tokens were then discovered by the transformer from scratch by looking at the data.

Transformer's memory usage increases with O(n²) where n is the length of input sequence. When n is smaller than a few hundreds, a transformer model is trainable with a local GPU, but beyond that is not practical or feasible. To circumvent this issue while exploiting the capabilities of the transformer, the SAN implementation structures input data into three levels of a hierarchy (see FIG. 2). In level 1 (L1), a transformer learned to encode each individual MS/MS spectrum. In level 2, another transformer learned to encode a sequence of spectra, i.e. a sequence of L1 outputs. In level 2.5, a simple linear classifier learned to classify disease status (as cancerous or disease free) from a sequence of L2 outputs.

### Self-Supervised Training

To make up for a low example count, self-supervised training was extensively used. In L1, model was asked to guess masked token, similar to masked language modeling in NLP. In the level 2 (L2), model was asked to predict masked inputs.

The algorithm components described above focused to learn and encode input sequence position-by-position and thus the output at a specific position is specific to the very position. This is a limitation to understand a broad context about the input. To encourage the model to learn higher level features, a secondary objective was used. Similar to the next-sentence-prediction task in NLP, where a model is asked to predict whether two sentences are related or not, the L1 model was asked to predict whether two spectra are adjacent. In L2, the model was asked to predict whether two sequences of spectra are from the same sample.

### Implementation Details

A Pancreatic cancer data-set was used containing 118 raw mass spec files collected from the same number of samples. The gradient length of the LC-MS used to collect the files was 180 mins resulting in approximately 231K spectra across 70 isolation windows. Raw files were converted to mzml using msconvert. For the conversion, cwt peak peaking is selected. Mz and intensity were written as single precision floats. Also, zero samples (zero intensity peaks) were removed. The full conversion was performed using the following msconvert settings “--64--mz32--inten32--filter “peakPicking cwt” --filter “zeroSamples removeExtra”” The dataset was stratified-split into 80% train, 10% validation, and 10% test set using healthy vs. cancer label by sklearn library.

### Tokenization

A Tokenization process converted mzml files to python pickle files. The pickle file contained a list, whose element is a dict. The dict had one key token and value contained a list of numpy arrays. The numpy array had the token id of each spectrum. The pickle object was referenced by data[window_idx][‘token’][spectrum_idx][peak_idx]. The following steps were then performed:
Search for MS level 2 spectrum.
Sort peaks in intensity descending order.
Take top 150 peaks and discard the rest.
Multiply mz value by 0.9995.
clip mz value to [100, 1800].
Round mz value to the nearest integer.
Add offset −90=10 for reserved tokens−100 for min mz value.
Trim to Remove head and tail of experiment ⅙ each and ⅓ total, Reducing the number of spectrum per isolation window from 3.3K to 2.2K.

### Encoder Level 1

A transformer encoder was trained with two tasks—masked token prediction and adjacency prediction. Two cross-entropy losses were added with equal weights. Adjacency was labeled into three classes—unrelated, adjacent in horizontal(time) axis, and adjacent in vertical(isolation window) axis.

Target adjacency label was sampled with the equal weights. Using the target label, two spectra were sampled from dataset. One summary token, and two spectra are concatenated. 10% of tokens from each spectrum is masked. Token type id is set to 0 for the summary token and the first spectrum. The second spectrum has token type id of 1. Token embedding is followed by layer norm and dropout. The transformer block was configured to have 6 layers, 512 hidden dimensions, 3*512 intermediate dimensions, 8 attention heads, absolute position encoding. Token prediction head was dot product with token embedding table, followed by a bias layer. Linear head was used for adjacency prediction.

Training was performed using AdamW with learning rate of 2e-4 and weight decay of 4e-5. Beta1=0.9, Beta2=0.95. Embedding, bias, and layer norm parameters were excluded from weight decay. Learning rate was cosine decayed with alpha=0.1; Batch=512. g5x instance with 4 GPUs; Epoch steps=(106*70*2200//BATCH_L1//10); Target epochs=100. Training of the encoder took 1-2 days.

Pre-encoding of Level 1: After level 1 training was finished, level 1 transformer was fixed and dataset was encoded using the transformer. As level 1 is trained using two spectra, two spectra are fed to the encoder and output of adjacency prediction token is taken. This reduced the sequence length of isolation window 2.2K to 1.1K. After pre-encoding, two spectra were represented by one 512 dimensional vector.

### Encoder Level 2

Level 2 was constructed similar to level 1. The core difference is that the input element was already in a high dimensional vector, thus token embedding was not needed. Masked token prediction in level 1 was replaced with masked vector prediction. Adjacency prediction was replaced with inter-intra person prediction.

Masked vector prediction: The masked input vector was replaced by a learnable vector. Output of the encoder was dot-producted with the original contents of the vector. Cross-entropy loss was used. In level 1, dot-product was taken across token-space, i.e. total ~2000 categories. In level 2, dot-product was taken across masked inputs including the ones in batch. As the batch size gets larger, the difficulty of the task increases, as does the loss of quality.

Inter-intra person prediction: Given two sequences of vectors from two isolation windows, the model was asked to guess whether two sequences stem from one person or two persons.

Target inter-intra person prediction label was sampled randomly with equal weights. Two isolation windows were selected and random jitter amount (~3% of sequence length) was added as offset to read from. 10% of the input was masked.

Every three input elements were grouped together and go through fully connected layer to produce one 512-dimensional vector. This reduced the sequence length of one isolation window from 1.1K to ~350. The Transformer block was similar to level 1, except the number of layer is increased to 9 from 6.

Training required about 2 days on a local machine using the following settings: Batch=24; Epoch steps=(10*70*116)//BATCH_L2); Target epochs=100;

### Classifier (Level 2.5)

The level 2 outputs, from all 70 isolation windows, were fed to linear classifier. The Level 2 encoder and linear classifier were trained together.

Dataset: Only 2 examples can be fit in 48G memory at one time. Random sampling one positive and one negative example to bundle two as one mini batch was used.

Training setting for the linear classifier were: LR=2e-4, WD=1e-3; Batch=2; Epoch steps=1*94//BATCH_FINE_TUNE_AGGREGATE; Target epoch=10

Evaluation on validation split and test split were performed.

### Example 3 Discovery of New Condition Markers

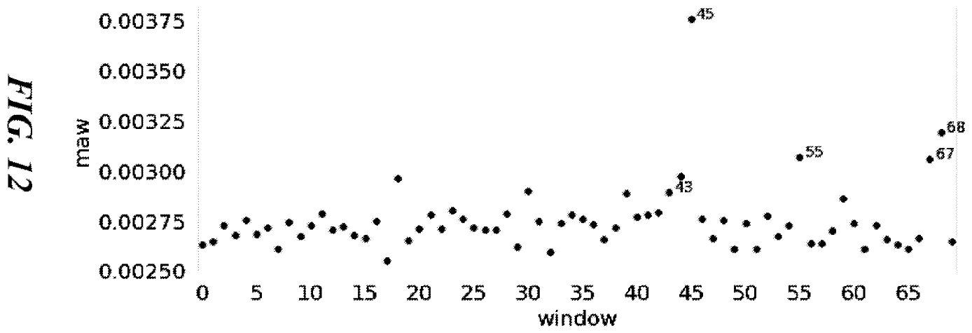

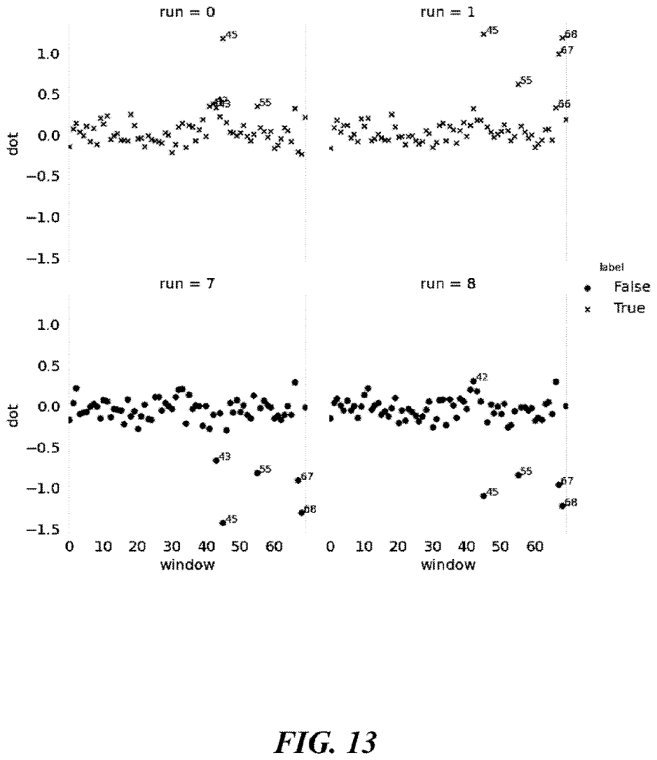

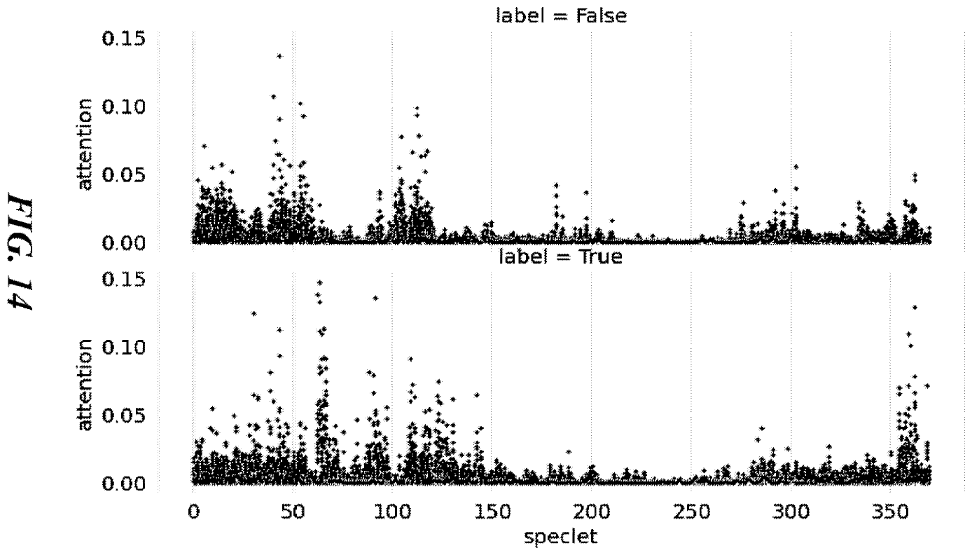

Model parameters were extracted from a model trained to classify whether or not a subject has cancer. Results are shown in FIGS. 12-14. Extraction of the model parameters allow for identification of biomarkers which are indicative of either a heathy subject or a subject with cancer.

### Example 4 Protein Discovery and Disease Classification Using Alternate Hierarchical Transformers

An alternate hierarchy implementations of the tokenization, hierarchy, and self-supervised training methods described herein was developed as follows for DIA-mode experiments:

Since one typical DIA mode experiment contains more than tens of thousands of spectra including such data in a transformer model is challenging. The architecture of Examples 2 and 3 address this concern by breaking down the data into two levels—spectrum level (1st) and isolation window level (2nd)—so that the data size becomes manageable by the transformer model.

The number of examples in a typical clinical study of a condition of a subject is on the order of patient samples times isolation windows, which is less than 10K sample points for a typical dataset that can be used to train the second level of the hierarchy. Additionally, the level-1 model is frozen after pre-training, is not updated during fine-tuning stage, and does not receive or utilize any label information.

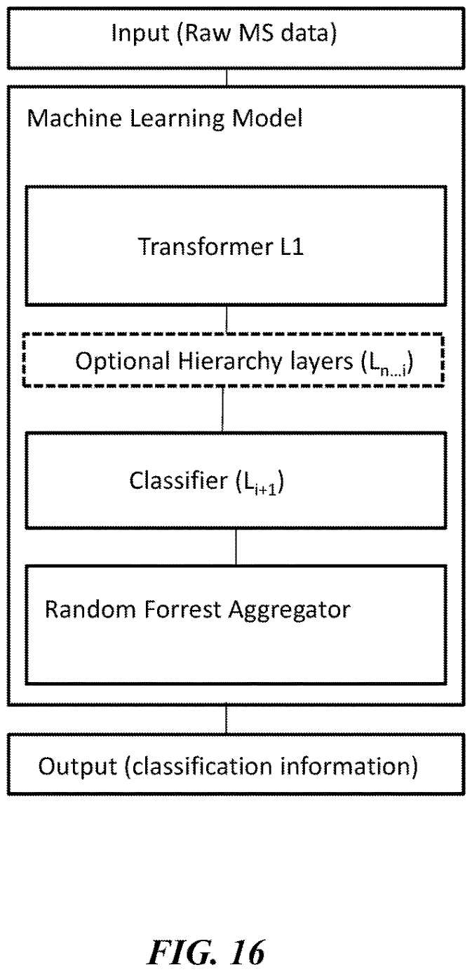

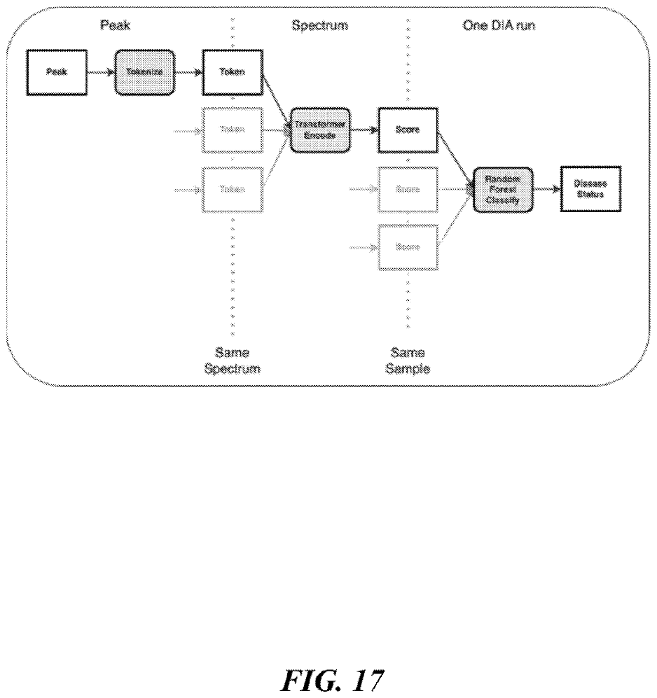

To improve the number of examples which are available in certain applications to train such a two level model, and to increase the fidelity of both levels of the model, an alternate hierarchy was developed. In the alternate hierarchy, the Level-2 model and subsequent linear classifier are substituted for a direct linear classifier, which produces a score directly. The score indicates how likely the given spectrum belongs to a sample of label 0 or 1. The hierarchy is trained with 10K+ spectra and their scores given one sample. A random forest model aggregates the scores from the linear classified and outputs the final score. A high-level overview of this workflow is presented in FIG. 16, and a more detailed diagram of the Example scheme is presented in FIG. 17.

The transformer encoder learns spectrum level information, allowing for direct fine-tuning, which means that label information is injected into the model. The use of random forest as information aggregator facilitates back-tracing of results. For example, inspecting feature importance of the model can reveal which spectrum is important and contributes to the final output the most.

Also given an important spectrum, it is possible to inspect an attention matrix of the transformer model to see which peak contributed to the score more.

Among 10K+ spectra in a sample, only small fraction of spectra are important or have mutual information with label. Beforehand, we don't know which are important and train with all spectra. Examples with low quality or signal-to-noise ratio can have a negative impact on training. Two different fine-tuning schemes implemented in this example are designed to alleviate this problem, as described below in the fine-tuning section.

A random forest model treats each score as a unique feature. If one sample has retention time offset relative to other samples, spectra and their scores will propagate as an offset in the feature space of the random forest. A simple retention time alignment step was added to cancel out retention drift as much as possible, as illustrated in the dataset section below.

Some implementations of the Example scheme included a transformer model that takes multiple spectra as input, which can absorb some of the remaining offset in the input, as described in the model section below.

### Example 4 Implementation Details:

Dataset preparation: Raw data files were centroided and deisotoped.

Tokenization was performed substantially as described in Example 2.

A trim step was extended to compensate retention time offset by calculating an offset for each sample, which is added to the trimming range.

### Offset Calculation:

A simple data-driven method was used as follows: Each DIA run was converted to 3D matrix whose axis was isolation window index, retention time index, and binned mz index. The value was log of peak intensity or 0 if there is no peak in the given index.

For each matrix, retention time was adjusted with an offset to maximize sum of similarity to other matrix. This was performed in a loop until the offset values converged.

### Model:

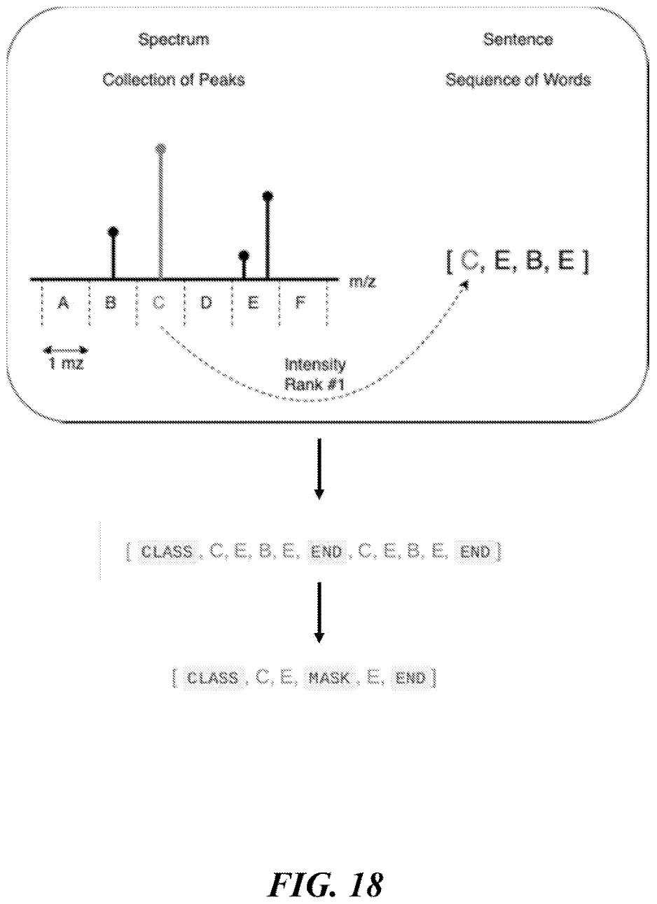

The basic principle of converting spectrum into sequence was performed substantially as in Example 3. Each peak was sorted by intensity rank order and mapped to a fixed sized m/z bin. An example of this is illustrated in FIG. 18. In addition to the converted sequence, classification token is prepended. The token encourages the model to summarize the information of the whole spectrum. Its encoded output is fed to a linear classifier.

The Example model was capable of handling more than one spectrum at a time, which can extract more information from time-adjacent, multiple spectra. Multiple sequences from multiple spectra are concatenated to form one sequence, with END token inserted between.

For example, given two spectra that are identical to diagram in FIG. 18, the converted sequence will be: [CLASS, C, E, B, E, END, C, E, B, E, END].

The number of spectra to combine is a hyper-parameter. Different values have been tried and evaluated, ranging from 1 to 6.

### Pre-Training:

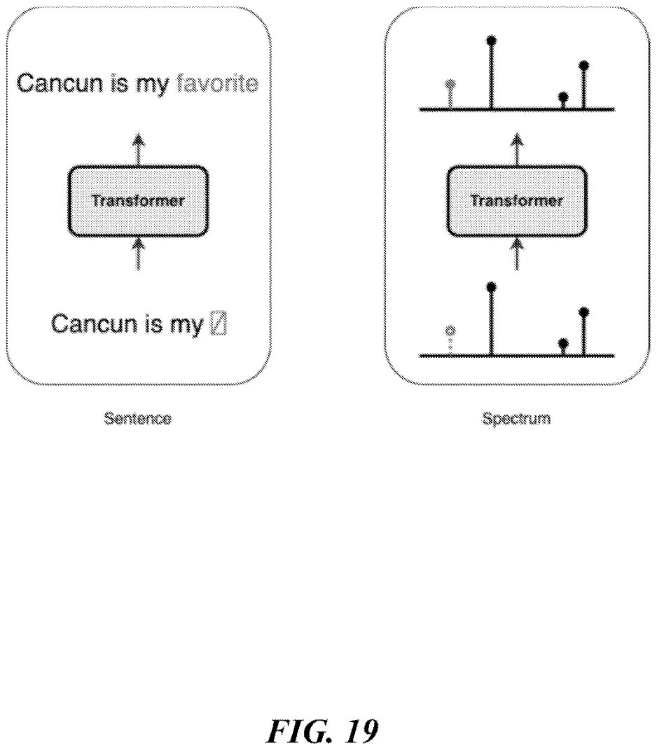

In pre-training step, 15% of tokens are randomly hidden, similar to masked language model training. The diagram illustrated in FIG. 19 shows conceptual analogy between sentence and spectrum pre-training. Putting the spectrum example above In sequence form: [CLASS, C, E, MASK, E, END], the model is trained to predict MASK token with expected golden answer to be B.

### Fine-Tuning:

The Example model learns the general distribution of input data during pre-training step. In fine-tuning step, the model learns how label is related to the input and is trained to predict the label given the input. To do that, the output of CLASS token is fed to a linear classifier that predicts the label.

### Global Scheme:

Straightforward application of fine-tuning is:
Initialize model from pre-trained model.
Sample train example spectrum evenly from dataset.
Train the model with the example and label.

### Hyper-Local Scheme:

Instead of having one model trained with all spectra in the dataset, a hyper-local scheme builds and fine-tune model separately for each isolation and retention time index:
Loop for each isolation and retention index.
Initialize model from pre-trained model.
Pick example spectrum from the specific index.
Train the model with the example and label.
   Global vs. Hyper-Local:

In the global scheme, the example model is trained with more diverse spectra. However, only small portion of spectra that comes from specific isolation window and retention time have mutual information with label. Other spectra can be low quality examples and feeding them might have negative impact.

In hyper-local scheme, the example model is fine-tuned with spectra from a specific isolation window and retention time. This prevents high fidelity information examples from being mixed with low fidelity information examples. However, the number of training examples used can be significantly smaller.

Both schemes were evaluated in models as described in Example 4. Hyper-local scheme performed slightly better and is discussed in detail for the results section below.

### Results:

Due to the nature of deep learning, it hard to interpret and understand output of model. Even if evaluation metrics are good, it is not always obvious why a model made a particular decision or based on what features. To thoroughly evaluate the example models in a controlled fashion, a problem with known biomarker proteins was selected as a test case to allow the important spectra that are found by model to be compared against known proteins and their spectra. The results of these test cases are described below:

Plasma vs. Serum: Compared to plasma blood sample, serum goes through extra processing. Fibrinogen protein is known to be filtered out. A SAN model as described above was trained with a dataset of mixed plasma and serum samples. A label was assigned to indicate whether the sample is plasma or serum.

The accuracy and AUC for the test split was around 0.99.

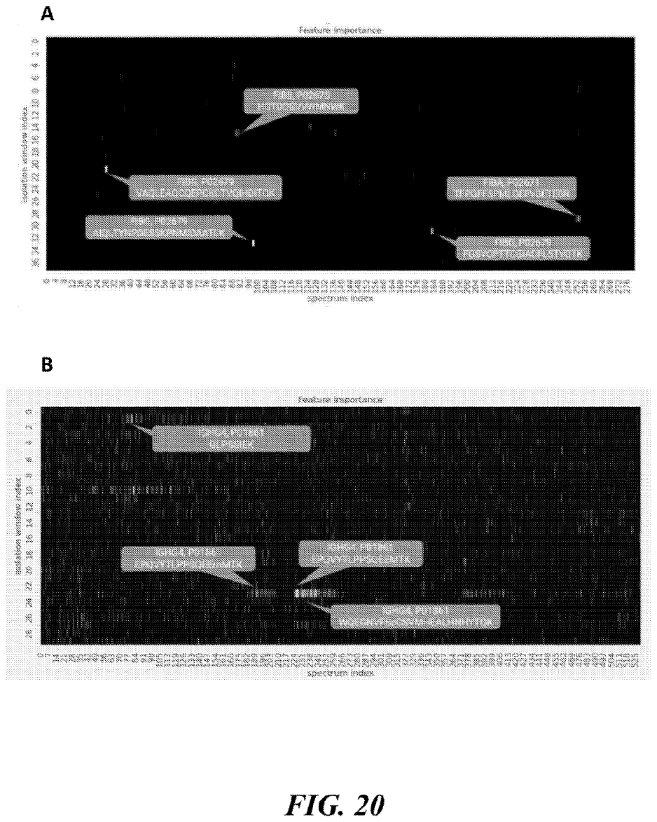

The FIG. 20A shows feature importance of random forest classifier. The random forest model treats each isolation window index and spectrum(retention time) index as a separate input feature. Among thousands of such features, the model finds important ones that are helpful to predict the label. Important features were compared against a list of peptides in fibrinogen alpha and gamma protein. The list is produced by DIA-NN tool and has isolation window and retention time information. The FIG. 20A shows that SAN feature list overlaps with the peptide list from DIA-NN tool.

A more challenging test case was created using quantification results from DIA-NN. A single protein was handpicked and a label was assigned based on the quantity of the protein—label 1 for high quantity and 0 for low quantity.

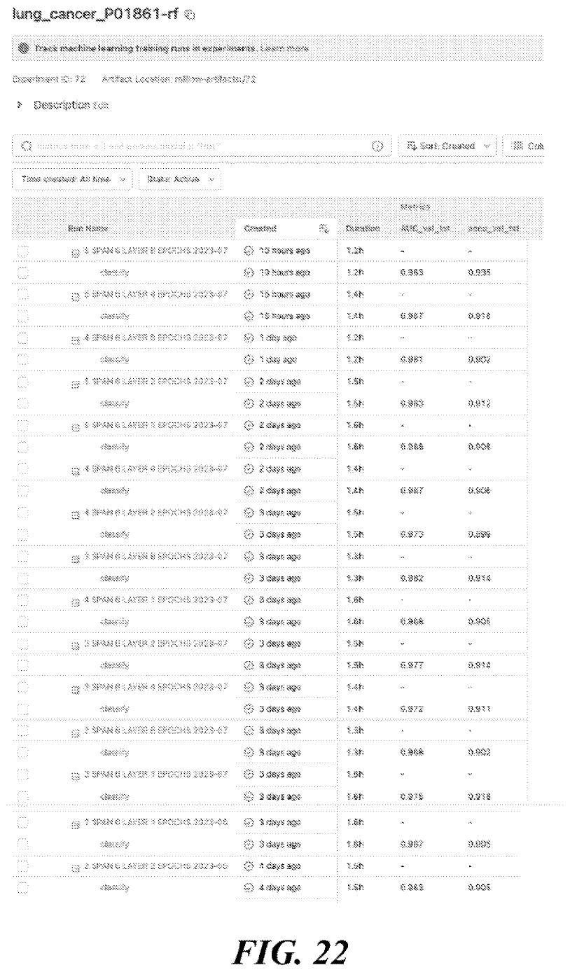

Different variant of models achieved around 0.95 AUC and 0.9 accuracy, as illustrated in FIG. 22. FIG. 20B shows that a few top features overlap with DIA-NN peptide list.

Another protein, P08519, was chosen and repeated the same procedure as above.

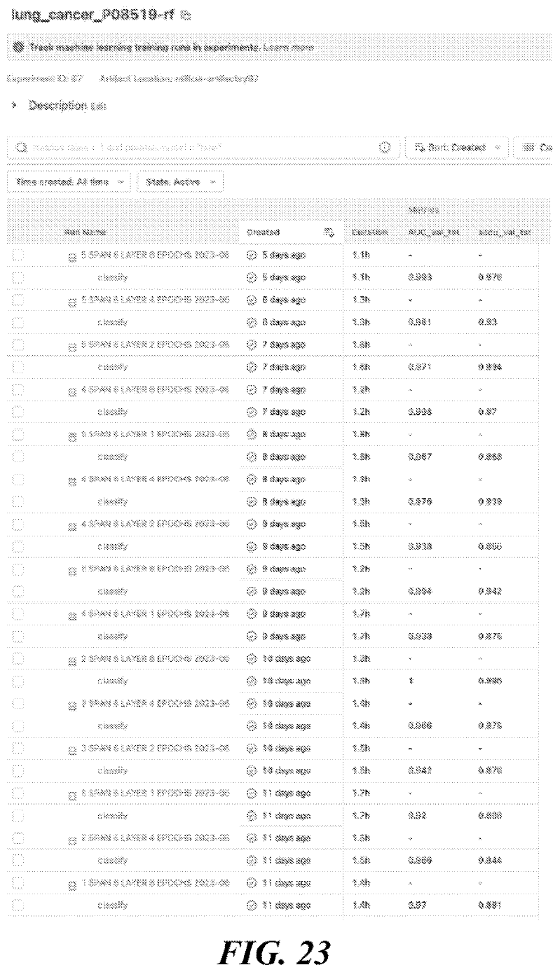

Different variant of models achieved around 0.95 AUC and 0.9 accuracy. The variance among models was higher than protein P01861, as shown in FIG. 23

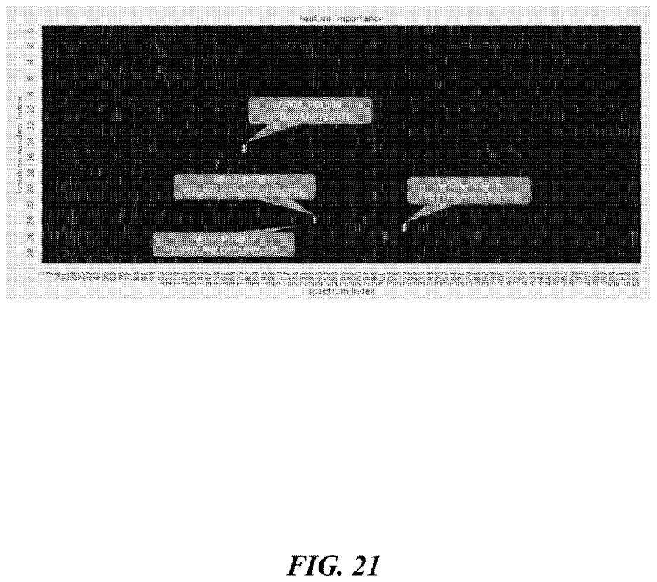

FIG. 21 shows that a few top features overlap with DIA-NN peptide list.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the present disclosure. Furthermore, it shall be understood that all aspects of the present disclosure are not limited to the specific depictions, configurations or relative proportions set forth herein which depend upon a variety of conditions and variables. It should be understood that various alternatives to the embodiments of the present disclosure described herein may be employed in practicing the present disclosure. It is therefore contemplated that the present disclosure shall also cover any such alternatives, modifications, variations, or equivalents. It is intended that the following claims define the scope of the present disclosure and that methods and structures within the scope of these claims and their equivalents be covered thereby.

What is claimed is:

1. A method of characterizing a condition of a subject using mass spectrometry data, the method comprising:
obtaining one or more raw mass spectra of a sample from the subject collected using a mass spectrometer, wherein the raw mass spectra comprises ion m/z values and intensities, wherein an experimental m/Δm resolving power of the mass spectrometer is about 500-2,000,000 at m/z 200;
providing a machine learning model comprising one or more transformers that are trained on a raw mass spectra training dataset for characterization of the condition of the subject; and
using the machine learning model to analyze the one or more raw mass spectra of the sample and output information indicative of at least one condition marker or condition state in the subject.

2. The method of claim 1, further comprising providing the information to a user via a graphical user interface.

3. The method of claim 1, wherein the experimental m/Δm resolving power is about 500-1,000,000 at m/z 200.

4. The method of any of claims 1-3, wherein the experimental m/Δm resolving power is about 500-30,000 at m/z 200.

5. The method of any of claims 1-4, wherein the experimental m/Δm resolving power is about 500-5,000 at m/z 200.

6. The method of any of the preceding claims, wherein the condition comprises a disease.

7. The method of any of the preceding claims, wherein the condition comprises an age state of the subject.

8. The method of any of the preceding claims, wherein the condition comprises a progression-free survival of the subject.

9. The method of any one of the preceding claims, wherein the machine learning model further comprises a linear classifier.

10. The method of any one of the preceding claims, wherein the machine learning model further comprises a neural radiance field.

11. The method of any one of the preceding claims, wherein the machine learning model further comprises a multi-layer neural network.

12. The method of any one of the preceding claims, wherein the machine learning model further comprises a decision tree.

13. The method of any one of the preceding claims, wherein the machine learning model further comprises a support vector machine.

14. The method of any one of the preceding claims, wherein the one or more raw mass spectra comprise MS/MS spectra.

15. The method of claim 14, wherein the MS/MS spectra are acquired in a data independent manner.

16. The method of claim any of the preceding claims, wherein the machine learning model comprises a plurality of transformers.

17. The method of claim 16, wherein the plurality of transformers are arranged in a hierarchy comprising a first and second transformer arranged in a hierarchy such that an output of the first transformer is used as an input of the second transformer.

18. The method of any one of the preceding claims, wherein the one or more raw mass spectra are tokenized prior to submission to the one or more transformers.

19. The method of any one of the preceding claims, wherein the machine learning model is trained with at least 10,000 individual mass spectra per day.

20. The method of any of the preceding claims, wherein the machine learning model is trained with at least 50,000 individual mass spectra per day.

21. The method of any of the preceding claims, wherein the machine learning model is trained with at least 100,000 individual mass spectra per day.

22. A machine-learning based method of characterizing a condition of a subject, the method comprising:
obtaining one or more raw mass spectra of a sample from the subject collected using a mass spectrometer;
providing a machine learning model comprising a plurality of transformers that are arranged in a hierarchy and trained on a raw mass spectra training dataset for characterization of the condition; and
using the machine learning model to analyze the one or more raw mass spectra of the sample and output information indicative of at least one condition or condition state in the subject.

23. The method of claim 22, wherein the hierarchy comprises a first and second transformer in a hierarchy such that an output of the first transformer is used as an input of the second transformer.

24. The method of claim 23, further wherein the hierarchy further comprises a linear classifier, the linear model being arranged in the hierarchy such that an output of the second transformer is used as an input of the linear classifier.

25. The method of claim 23, further wherein the hierarchy further comprises a neural radiance field, the neural radiance field being arranged in the hierarchy such that an output of the second transformer is used as an input of the neural radiance field.

26. The method of claim 23, further wherein the hierarchy further comprises a multi-layer neural network, the multi-layer neural network being arranged in the hierarchy such that an output of the second transformer is used as an input of the multi-layer neural network.

27. The method of claim 23, further wherein the hierarchy further comprises a decision tree, the decision tree being arranged in the hierarchy such that an output of the second transformer is used as an input of the decision tree.

28. The method of claim 23, further wherein the hierarchy further comprises a support vector machine, the support vector machine being arranged in the hierarchy such that an output of the second transformer is used as an input of the support vector machine.

29. The method of any one of claims 24-28, wherein the first transformer classifies tokenized data based on an MS/MS isolation window.

30. The method of claim 29, wherein the second transformer classifies a vector output of the first transformer based upon a sample identity.

31. The method of claim 30, wherein the linear classifier classifies the disease or disease state based on the vector output from the second transformer.

32. The method of any one of the preceding claims, wherein the raw mass spectra comprise MS/MS spectra that are acquired in a data independent manner.

33. The method of any one of the preceding claims, wherein the machine learning model is trained with at least 10,000 individual mass spectra per day.

34. The method of any of the preceding claims, wherein the machine learning model is trained with at least 50,000 individual mass spectra per day.

35. The method of any of the preceding claims, wherein the machine learning model is trained with at least 100,000 individual mass spectra per day.

36. The method of any of the preceding claims, wherein the condition comprises a disease.

37. The method of any of the preceding claims, wherein the condition comprises an age state of the subject.

38. The method of any of the preceding claims, wherein the condition comprises a progression-free survival of the subject.

39. A method of characterizing a condition of a subject using a high throughput trained machine learning model, the method comprising:
obtaining one or more raw mass spectra of a sample from the subject collected using a mass spectrometer;
providing the machine learning model that is trained on a raw mass spectra training dataset for characterization of the condition, wherein the machine learning model is trained at a rate of at least 10,000 individual raw mass spectra from the training dataset per day; and
using the machine learning model to analyze the one or more raw mass spectra of the sample and output information indicative of at least one condition in the subject.

40. The method of claim 39, wherein the rate is at least 50,000 individual raw mass spectra from the training set per day.

41. The method of claim 39, wherein the rate is at least 100,000 individual raw mass spectra from the training set per day.

42. The method of any one of the preceding claims, wherein the machine learning model further comprises a linear classifier.

43. The method of any one of the preceding claims, wherein the one or more raw mass spectra comprise MS/MS spectra.

44. The method of any of the preceding claims, wherein the machine learning model comprises a plurality of transformers.

45. The method of claim 44, wherein the plurality of transformers are arranged in a hierarchy comprising a first and second transformer in a hierarchy arranged such that an output of the first transformer is used as an input of the second transformer.

46. The method of any one of the preceding claims, wherein the one or more raw mass spectra are tokenized prior to submission to the one or more transformers.

47. The method of any of the preceding claims, wherein the condition comprises a disease.

48. The method of any of the preceding claims, wherein the condition comprises an age state of the subject.

49. The method of any of the preceding claims, wherein the condition comprises a progression-free survival of the subject.

50. The method of any one of the preceding claims, wherein the one or more raw mass spectra are tokenized by an MS/MS isolation window and a plurality of m/z values corresponding to detected ions of each of the one or more raw mass spectra.

51. The method of claim 50, wherein the one or more raw mass spectra are tokenized such that m/z values with the same unit mass are binned together.

52. The method of claim 50, wherein the one or more raw mass spectra are tokenized using large bins (e.g. bins spanning about 1, 0.7, 0.5, or 0.3 mass units).

53. The method of claim 50, wherein the one or more raw mass spectra are tokenized using small bins (e.g. bins spanning about 0.1, 0.01, 0.001 or less mass units).

54. The method of claim 50, wherein the one or more raw mass spectra are tokenized using uniform bins.

55. The method of claim 50, wherein the one or more raw mass spectra are tokenized using non-uniform bins.

56. The method of any of the preceding claims, wherein the machine learning model is trained using self-supervised learning.

57. The method of any one of the preceding claims, wherein measuring the sample by mass spectrometry comprises separating components of the sample using liquid chromatography coupled to a mass spectrometer.

58. The method of claim 57, wherein a gradient method of the liquid chromatography runs over a period of at least 15 minutes (e.g. about 15, 30, 60, 90, or 180 minutes).

59. The method of claim 57, wherein a gradient method of the liquid chromatography runs over a period of about 5 to 10 minutes (e.g. about 5, 7, or 10 minutes).

60. The method of any of the preceding claims, wherein the information includes presence or absence of the at least one disease or disease state in the subject.

61. The method of any of the preceding claims, wherein the at least one disease or disease state comprises cancer.

62. The method of claim 61, wherein the cancer comprises pancreatic cancer or ovarian cancer.

63. The method of claim 61, wherein the cancer comprises breast cancer.

64. The method of claim 61, wherein the cancer comprises prostate cancer.

65. The method of claim 61, wherein the cancer comprises lung cancer.

66. The method of claim 61, wherein the cancer comprises gallbladder cancer.

67. The method of any of the preceding claims, wherein the condition comprises a plurality of disease states.

68. The method of any of the preceding claims, wherein the condition is a disease state, and the disease state comprises a responsiveness of a disease to a therapeutic intervention.

69. The method of claim 68, wherein the therapeutic intervention is an immunotherapy (e.g. a CAR-T therapy).

70. The method of any of the preceding claims, wherein the information comprises a probability or likelihood of the subject having the at least one disease or disease state.

71. The method of any of the preceding claims, wherein the information comprises an indication of disease state or disease severity.

72. The method of any of the preceding claims, wherein the information comprises an indication of disease classification.

73. The method of claim 72, wherein the at least one disease or disease state is a cancer and the indication of the disease classification comprises an identification of a cell line genotype or cell line phenotype of the cancer.

74. The method of any one of the preceding claims, wherein the information is associated with at least one of a proteomic, a lipidomic, or a metabolomic profile of the sample obtained from the subject.

75. The method of claim 74, wherein the machine learning model outputs the information without requiring prior domain knowledge relating to at least one of the proteomic, lipidomic, or metabolomic profile.

76. The method of any one of the preceding claims, wherein an accuracy of the information is at least 70%.

77. The method of any one of the preceding claims, wherein an accuracy of the information is at least 80%.

78. The method of any one of the preceding claims, wherein an accuracy of the information is at least 90%.

79. The method of any one of the preceding claims, wherein an accuracy of the information is at least 95%.

80. The method of any one of the preceding claims, wherein an accuracy of the information is at least 99%.

81. The method of any one of the preceding claims, wherein training the machine learning model to determine a presence or absence of the one or more disease conditions requires no more than about 500 experimental data points.

82. The method of claim 81, wherein no more than about 200 experimental data points are required to train the machine learning model.

83. The method of claim 81, wherein no more than about 100 experimental data points are required to train the machine learning model.

84. The method of claim 81, wherein an accuracy of the determination is at least about 70%.

85. The method of claim 74, wherein the proteomic profile comprises one or more post-translational modifications (PTMs).

86. The method of claim 85, wherein the post-translational modifications comprise one or more phosphorylation, acetylation, ubiquitination, glycosylation, or combination of two or more thereof.

87. The method of any one of the preceding claims, wherein the training the machine learning model comprises randomly masking about 1-25% (e.g. 1%, 5%, 10%, 15%, 20%, or 25%) of the training set and adding about 1-10% (e.g. about 1%, 2%, 3%, 4%, 5%, or 10%) noise as a means of self-supervised learning.

88. The method of any one of the preceding claims, wherein measuring the sample by mass spectrometry comprises separating ions by ion mobility (e.g. by High Field Asymmetric Waveform Ion Mobility Spectrometry (FAIMS) or Drift-tube Ion Mobility Spectrometry) prior to or during acquisition mass spectra.

89. The method of any one of the preceding claims, wherein a mean average percent error of the information is less than about 30% (e.g. less than 30%, 20%, 15%, 10%, 5%, 3%, 2%, or 1%).

90. The method of any of the preceding claims, wherein adjacent m/z values are not treated as continuous values during the analysis.

91. The method of any of the preceding claims, wherein the information comprises identification of one or more signal which is determinative of the presence or absence of a particular condition.

92. The method of any of the preceding claims, wherein the information comprises identification of one or more signal which is indicative or correlated with a particular state of a particular condition.

93. The method of any of the preceding claims, wherein the information is used for biomarker discovery.

94. The method of any of the preceding claims, further comprising raw mass spectra are converted to preprocessed mass spectra by an automated algorithm comprising a de-isotoping, a de-charging, or a de-adducting algorithm.

95. The method of any of the preceding claims, wherein the method is capable of being trained at a rate of at least 10 training samples per day (e.g at least 10, 15, 50, 100, 300, 500, or 700 samples per day) when trained using a single GPU or CPU which is no faster in terms of maximum single precision floating point operations per second than an NVidia RTX A6000 GPU equipped with 48 GB of RAM.

96. The method of any of the preceding claims, wherein the machine learning model comprises a transformer arranged in a hierarchy with a linear classifier and a random forest aggregator.

97. A non-transitory computer-readable storage medium comprising instructions that, when executed by a processor, causes the processor to perform the method of any of the preceding claims.

98. A system configured for characterizing a condition of a subject, the system comprising:
a computer comprising a memory operably coupled to at least one processor; and
a module executing in the memory of the computer, the module comprising program code enabled upon execution by the at least one processor of the computer to perform:
the method of any of the preceding claims.
